(19) United States
(12) Patent Application Publication
(10) Pub. No.: US 2025/0332008 A1
(43) Pub. Date: Oct. 30, 2025

(54) RESHAPING A BLOOD VESSEL FOR IMPROVING BLOOD CIRCULATION

(71) Applicant: EDWARDS LIFESCIENCES CORPORATION, Irvine, CA (US)

(72) Inventors: Gideon Meyer-Brodnitz, Ramot Menashe (IL); Ben Shitrit, Eilat (IL); Gil Senesh, Adi (IL); Roei Mayo, Irvine, CA (US); Yuval Kasher, Kfar Shmuel (IL)

(21) Appl. No.: 19/264,710

(22) Filed: Jul. 9, 2025

Related U.S. Application Data
(63) Continuation of application No. PCT/US2024/012834, filed on Jan. 24, 2024.
(60) Provisional application No. 63/481,978, filed on Jan. 27, 2023.

(51) Int. Cl. A61F 2/848

(52) U.S. Cl. CPC A61F 2/848; A61F 2002/8483; A61F 2220/0016; A61F 2230/0006; A61F 2230/0091; A61F 2250/0039

(57) ABSTRACT

Methods of reshaping blood vessels for improving compliance and increasing blood flow are disclosed. One preferred method includes deploying a tubular stent frame within a blood vessel. The stent frame preferably has a non-circular cross-section with a major-axis and a minor-axis. At least one tissue anchor is provided along a wall portion on the minor-axis. The tissue anchor is embedded into a wall of the blood vessel and the wall of the blood vessel is drawn into contact with the minor-axis wall portion. Preferably, tissue anchors are provided along opposing minor-axes of the stent frame for reshaping the blood vessel into a substantially oval cross-section. When exposed to high blood pressure, the stent frame expands to a more circular shape for allowing more blood to pass therethrough. Under low pressure, the stent frame returns to a non-circular cross-section to aid with the pumping of blood.

### RELATED APPLICATION(S)

This application is a continuation of International Patent Application No. PCT/US24/12834, filed Jan. 24, 2024, which claims the benefit of U.S. Provisional Patent Application No. 63/481,978, filed on Jan. 27, 2023, the complete disclosures of which are hereby incorporated by reference in their entireties.

### BACKGROUND

The present disclosure generally relates to the field of medical implant devices, including stent implant devices. Stent implant devices can be designed for intravascular deployment. Blood stagnation between stent implant devices and blood vessel tissue, such as in gaps formed between stent frames and blood vessel walls, can affect patient outcomes.

### SUMMARY

Described herein are devices, methods, and systems relating to non-circular stent devices/assemblies including tissue-engagement features/elements configured to couple one or more portions/aspects of a stent implant device to a surrounding blood vessel wall, which can advantageously facilitate physical coupling between the implant and the anatomy. Tissue-engagement elements can comprise barbs, hooks, spikes, or the like, and/or helical coil anchors or other tissue anchors. Tissue-engagement elements of stent examples of the present disclosure can be integrated with the stent implant, such as with the frame (e.g., metal frame) of the stent, or may be deployed and/or coupled in/to the stent implant in vivo. Stent-tissue coupling tissue-engagement features can be particularly useful with respect to stents that have medial portions that have a different, non-circular, cross-sectional shape compared to axial end portions thereof. For example, end portions of a stent may have generally circular axial cross-sectional shape, which may facilitate fluid sealing around the stent, while medial portion(s) of the stent can have a more-ovalized shape, which may facilitate cyclical stent reshaping to improve blood flow characteristics, as disclosed in detail herein.

For stent implant devices that include fluid-tight coverings, tissue-coupling features as disclosed herein can reduce the risk of blood stagnation radially outside of the stent. For stent implant devices that do not include fluid-tight coverings, tissue-coupling features as disclosed herein can couple the stent frame to the blood vessel wall in a manner as to allow for the blood vessel wall to serve as a flood flow channel that conforms to the shape/form of the stent frame.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Any of the example methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 shows example cardiac and vascular anatomy.

FIGS. 2A and 2B show side and axial cross-sectional views, respectively, of a compliant blood vessel experiencing compliant expansion and contraction over a cardiac cycle.

FIG. 3 shows an example stiff aorta.

FIGS. 4-1 and 4-2 show a blood vessel in circular and non-circular axial cross-sectional shapes, respectively.

FIGS. 5A and 5B show perspective and axial views, respectively, of a non-circular stent disposed in a blood vessel.

FIGS. 6A and 6B show perspective and axial views, respectively, of a non-circular stent disposed in a blood vessel in accordance with one or more examples.

FIGS. 7A-7D show perspective, side, and axial views, respectively, of a stent having circular end portions and a non-circular medial portion in accordance with one or more examples.

FIGS. 8A-8D show perspective, side, and axial views, respectively, of a stent having circular end portions and a non-circular medial portion in accordance with one or more examples.

FIGS. 9A and 9B show perspective and axial views, respectively, of a stent having oval (non-peanut) end portions and a peanut-shaped medial portion in accordance with one or more examples.

FIGS. 10A and 10B show side and axial views, respectively, of a stent implant device implanted within a blood vessel with trapped blood collected around portion(s) of the stent implant in accordance with one or more examples.

FIG. 11 shows a non-circular stent with tissue-engagement features in accordance with one or more examples.

FIGS. 12A and 12B show a non-circular stent with tissue-engagement features disposed within a blood vessel with a non-circular portion thereof circularized to engage the tissue-engagement features with the blood vessel wall in accordance with one or more examples.

FIGS. 13A and 13B show a non-circular stent disposed within a blood vessel with tissue-engagement features of a non-circular portion of the stent engaged with the blood vessel wall in accordance with one or more examples.

FIGS. 14-1, 14-2, 14-3, 14-4, and 14-5 illustrate a flow diagram for a process for engaging a non-circular portion of a stent with a blood vessel wall in accordance with one or more examples.

FIGS. 15-1, 15-2, 15-3, 15-4, 15-5, 15-6, 15-7, 15-8, 15-9, 15-10, 15-11, 15-12, 15-13, and 15-14 provide images of aspects of examples of the stent, tissue anchors, delivery system components, and anatomy relating to operations of the process of FIGS. 14-1, 14-2, 14-3, 14-4, and 14-5.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Vascular Anatomy and Compliance

Certain examples are disclosed herein in the context of vascular implant devices, and in particular, implant devices comprising non-circular segments having tissue-engagement elements associated therewith, wherein such implant devices are implanted in the aorta. However, although certain principles disclosed herein may be particularly applicable to the anatomy of the aorta, it should be understood that stent implant devices having tissue-engagement elements in accordance with the present disclosure may be implanted in, or configured for implantation in, any suitable or desirable blood vessels or other anatomy, such as the inferior vena cava.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves.

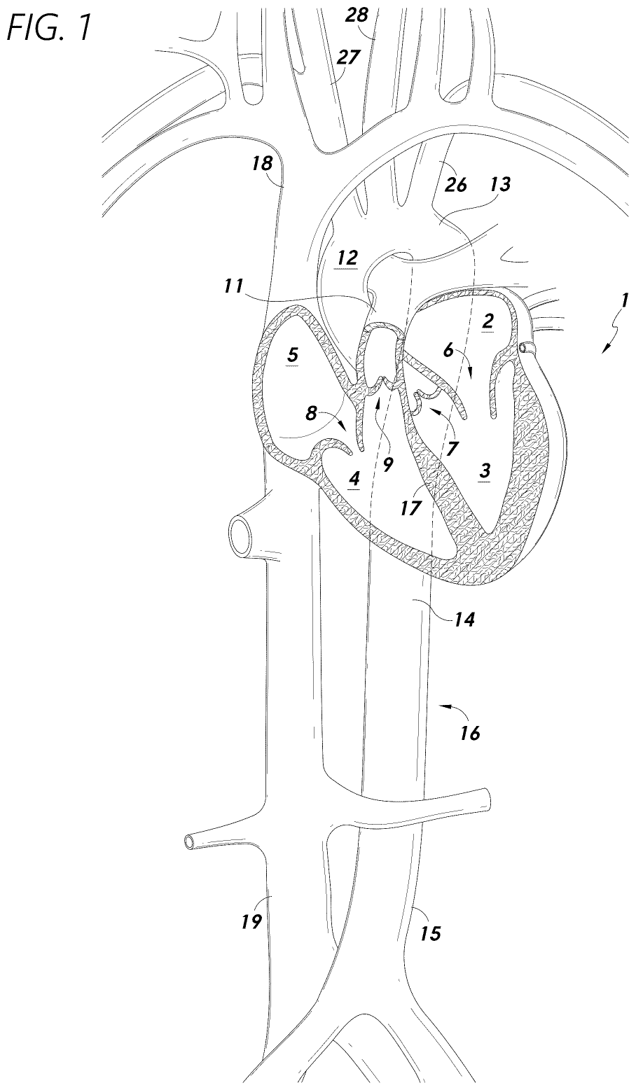

FIG. 1 illustrates an example representation of a heart 1 and associated vasculature having various features relevant to one or more examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left and right pulmonary arteries that branch off of the pulmonary trunk, as shown.

The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3. A wall of muscle 17, referred to as the septum, separates the left 2 and right 5 atria and the left 3 and right 4 ventricles.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta 16, carry blood away from the heart, whereas veins, such as the inferior 19 and superior 18 venae cavae, carry blood back to the heart.

The aorta 16 is a compliant arterial blood vessel that buffers and conducts pulsatile left ventricular output and contributes the largest component of total compliance of the arterial tree. The aorta 16 includes the ascending aorta 12, which begins at the opening of the aortic valve 7 in the left ventricle of the heart. The ascending aorta 12 and pulmonary trunk 11 twist around each other, causing the aorta 12 to start out posterior to the pulmonary trunk 11, but end by twisting to its right and anterior side. Among the various segments of the aorta 16, the ascending aorta 12 is relatively more frequently affected by aneurysms and dissections, often requiring open heart surgery to be repaired. The transition from ascending aorta 12 to aortic arch 13 is at the pericardial reflection on the aorta. At the root of the ascending aorta 12, the lumen has three small pockets between the cusps of the aortic valve and the wall of the aorta, which are called the aortic sinuses or the sinuses of Valsalva. The left aortic sinus contains the origin of the left coronary artery and the right aortic sinus likewise gives rise to the right coronary artery. Together, these two arteries supply the heart.

As mentioned above, the aorta 16 is coupled to the heart 1 via the aortic valve 7, which leads into the ascending aorta 12 and gives rise to the innominate artery 27, the left common carotid artery 28, and the left subclavian artery 26 along the aortic arch 13 before continuing as the descending thoracic aorta 14 and further the abdominal aorta 15. References herein to the aorta may be understood to refer to the ascending aorta 12 (also referred to as the “ascending thoracic aorta”), aortic arch 13, descending or thoracic aorta 14 (also referred to as the “descending thoracic aorta”), abdominal aorta 15, or other arterial blood vessel or portion thereof.

Arteries, such as the aorta 16, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

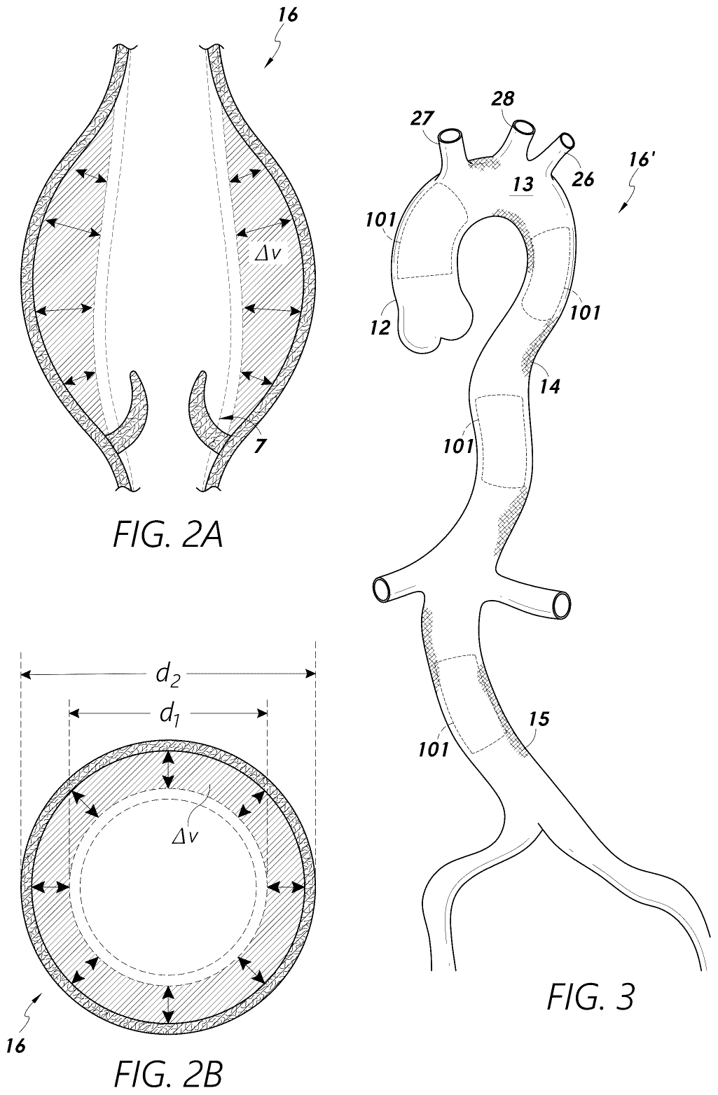

FIGS. 2A and 2B show side and axial cross-sectional views, respectively, of the healthy aorta 16 of FIG. 1 experiencing compliant expansion and contraction over a cardiac cycle.

As referenced above, the systolic phase of the cardiac cycle is associated with the pumping phase of the left ventricle, while the diastolic phase of the cardiac cycle is associated with the resting or filling phase of the left ventricle. As shown in FIGS. 2A and 2B, with proper arterial compliance, an increase in volume Δv will generally occur in an artery when the pressure in the artery is increased from diastole to systole. As blood is pumped into the aorta 16 through the aortic valve 7, the pressure in the aorta increases and the diameter of at least a portion thereof expands. A first portion of the blood entering the aorta 16 during systole may pass through the artery during the systolic phase, while a second portion (e.g., approximately half of the total blood volume) may be stored in the expanded volume Δv caused by compliant stretching of the blood vessel 16 from a non-expanded diameter d₁ to an expanded diameter d₂, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where Δv is the change in volume (e.g., in mL) of the blood vessel, and Δp is the pulse pressure from systole to diastole (e.g., in mmHg):

$$C = \frac{Δ v}{Δ p} \qquad ( 1 )$$

In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles and peripheral edema, for example, which may be caused by structural and/or functional cardiac abnormality. Such conditions can result in reduced cardiac output and/or elevated intra-cardiac pressures at rest or during stress.

FIG. 3 shows an example stiff aorta 16′. As shown in FIG. 3, the aorta tends to change in shape as a function of age, resulting in a higher degree of curvature and/or tortuosity over time. As the vasculature of a subject becomes less elastic, arterial blood pressure (e.g., left-ventricular afterload) becomes more pulsatile, which can have a deleterious effect. For example, undesirably pulsatile arterial blood flow, such as the thickening of the left ventricle muscle and/or diastolic heart failure. Stiffness in the aorta and/or other blood vessel(s) can occur due to an increase in collagen content and/or a corresponding decrease in elastin.

With the walls of the blood vessel 16′ being resistant to stretching due to the stiffness thereof, the expansion of the blood vessel diameter from the non-expanded diameter to the expanded diameter may be limited/reduced compared to the expansion of diameter of a healthy blood vessel. A stiff aorta 16′, as blood pressure increases, may experience a small amount of expansion and volume change, or the blood vessel may be sufficiently stiff that substantially no vessel expansion takes place during systole.

Generally, the majority of aortic compliance is provided in the ascending aorta 12 with respect to healthy anatomy. Furthermore, calcification frequently occurs in the area of the ascending aorta 12, near the aortic arch 13 and the great vessels emanating therefrom. Such anatomical areas can experience relatively higher stresses due to the geometry, elasticity, and flow dynamics associated therewith. Therefore, implantation/deployment of compliance-enhancing stent devices secured to blood vessel walls using circularizing support devices of the present disclosure can advantageously be in the ascending aorta 12 in some cases. While relatively less calcification tends to occur in the descending 14 and abdominal 15 aorta, implant devices of the present disclosure can advantageously be implanted/deployed in such areas as well for the purpose of increasing compliance in the aortic system. Examples of the present disclosure provide stent implant devices having non-circular frame segments including tissue-engagement elements, which may be implanted/secured within one or more locations in a compromised aorta and/or other vessel(s). For example, FIG. 3 shows example positions of stents 101 secured to blood vessel walls using features/aspects disclosed herein, such stents being implanted/disposed in various potential areas of the aorta 16′.

### Compliance-Enhancing Stent Implants

The present disclosure relates to delivery systems and methods for delivering various prosthetic implant devices in anatomy, such as vasculature, of a patient. As an example, implant devices that can be delivered using systems, devices, and methods disclosed herein can include stent or other implant devices configured to add-back and/or increase compliance in the aorta or other arterial (or venous) blood vessel(s) to provide improved perfusion of the heart muscle and/or other organ(s) of the body. For example, example stent implant devices of the present disclosure can include stents that, when implanted, are configured to decrease the cross-sectional area/volume of a target blood vessel segment in which the stent is implanted during low-pressure conditions, such as diastole, which serves to force blood through the blood vessel segment by pushing the blood through the vessel as the vessel volume reduces in connection with stent contraction induced by cyclical drops in blood pressure.

The non-circular (e.g., oval- and/or peanut-shaped) stents of the present disclosure can advantageously be configured to generate a differential cross-sectional area or volume of the target blood vessel(s) (e.g., aorta) between high- and low-pressure phases of the cardiac cycle to facilitate perfusion. As described above, relatively non-compliant blood vessels generally may not be able to stretch to thereby lengthen the perimeter of the blood vessel in response to increased pressure conditions. Such inability to stretch can prevent compliant expansion of the blood vessel.

Using non-circular stents to produce complaint blood vessel volume change by manipulating/reshaping the native blood vessel walls can increase compliance in a target blood vessel without requiring blood vessel grafting or resection. Therefore, compared to blood flow solutions involving blood vessel grafting/resection, non-circular stent examples of the present disclosure can provide solutions that avoid certain risks that may be associated with cutting of the vessel and/or devices grafted in/to such vessels, which may present risk of rupture and blood leakage outside of the circulatory system.

With respect to a blood vessel having a relatively fixed perimeter, wherein the blood vessel wall does not stretch/expand sufficiently due to stiffness and/or other factors of non-compliance, generally, the greatest area/volume of the blood vessel may be present/achieved when the blood vessel wall forms a circular cross-sectional shape. FIGS. 4-1 and 4-2 show a blood vessel in circular and non-circular axial cross-sectional shapes, respectively.

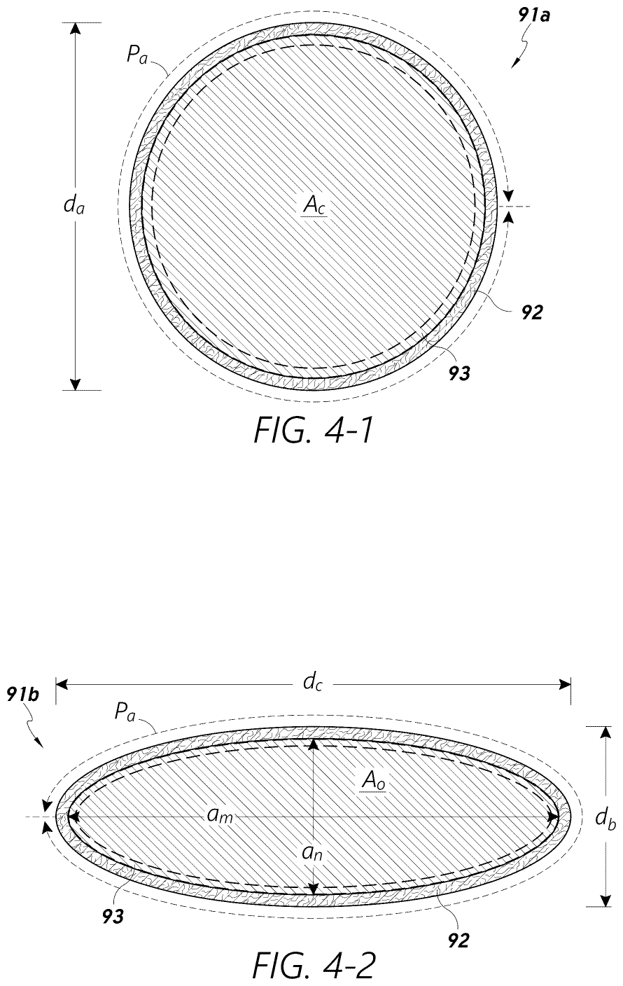

FIG. 4-1 shows an example blood vessel 91 (identified as blood vessel 91a in FIG. 4-1) having a generally circular cross-sectional shape, such that the area A_{c} thereof is maximized for the given perimeter/wall-length Pₐ. In the circular configuration, the diameter dₐ is substantially constant at every angle about the axis of the vessel. The circular shape of the vessel 91a may be set or permitted by the shape of a stent 93 implanted within the vessel.

Diverging from a circular cross-sectional shape can produce a cross-sectional area/volume for a blood vessel that is less than the maximum area A_{c} shown in FIG. 4-1. For example, FIG. 4-2 shows the blood vessel 91 (identified as vessel 91b in FIG. 4-2) having a shape that resembles an oval/ellipse, which produces the cross-sectional area Aₒ that is less than the area A_{c} with the same blood vessel wall/perimeter length Pₐ. The oval shape of the vessel 91b may have a major axis aₘ having a dimension d_{c} that is greater than a dimension d_{b} of the minor axis an thereof. The oval shape of the vessel 91b may be set/forced by the stent 93, which may have a biased oval shape.

With further reference to FIGS. 4-1 and 4-2, due to the area Aₒ of the oval vessel of FIG. 4-1 being less than the area A_{c} of the circular configuration shown in FIG. 4-1, transitioning from the circular shape 91a to the non-circular shape 91b, can provide a reduction in area/volume of the blood vessel, and therefore solutions that cause transitions between circular and non-circular blood vessel shapes between cardiac phases can provide compliance characteristics without the need for elasticity in the blood vessel wall tissue. For example, where a mechanism is implemented to cause a blood vessel to transition between circular and non-circular shapes in response to changing pressure conditions, such manipulation of the blood vessel shape can introduce volumetric change in the blood vessel in response to the typical changes in pressure experienced during the cardiac cycle, thereby increasing cardiac efficiency and reducing pulsatile load.

In view of the foregoing, examples of the present disclosure provide stent implant devices and associated processes configured to transition the shape/area of a blood vessel from circular/more-circular to non-circular/less-circular shapes, and vice versa, to enhance compliance with respect to the area of the implant reshaping. Such stent implant devices/processes may effect vessel reshaping through dynamic reshaping of the structural shape of the stent in a way that produces a change in shape of the blood vessel in which it is implanted to produce a change in blood vessel area/volume between the systolic and diastolic phases of the cardiac cycle. Tissue-engagement features, including spikes, barbs, and the like, as disclosed herein in association with stent frames, can increase the blood-vessel-reshaping capability of a stent implant, thereby further improving vascular compliance. The term “stent” is used herein in accordance with its broad and ordinary meaning and may refer to any device configured to be implanted in a lumen of a blood vessel, the device having a tubular form forming a lumen through which blood can flow.

Examples of the present disclosure provide for stent-type implants that are biased, with respect to at least a lengthwise portion/segment thereof, to a non-circular cross-sectional area, such that, in a relaxed/non-pressurized state, a first diameter of the stent has a greater dimension along a major axis compared to a second diameter of the stent along a minor axis, wherein such stents are configured to transition to a more-circular shape when pressure within the blood vessel overcomes the non-circular bias of the stent and causes the stent walls to be pushed to the more-circular configuration. The ability of stent implant devices of the present disclosure to reshape the target blood vessel in the manner described above to produce the desired oval cross-section of the blood vessel can be achievable due to stiff/non-compliant blood vessels, which may be unable to stretch to a substantial degree, still retaining the ability to bend/flex to a sufficient degree to allow for such shaping of the blood vessel. That is, the bending stiffness of a non-compliant blood vessel may be relatively lower compared to the stretching stiffness thereof. Therefore, examples of the present disclosure achieve compliance through bending energy with respect to the blood vessel wall, as opposed to stretching energy. When stents of the present disclosure are forced to a circular, or relatively more-circular, axial cross-sectional shapes, energy may be stored in the shape memory of the walls of the stent, wherein recoil/contraction of the stent towards its biased, oval/non-circular configuration can return/release energy to the blood circulation.

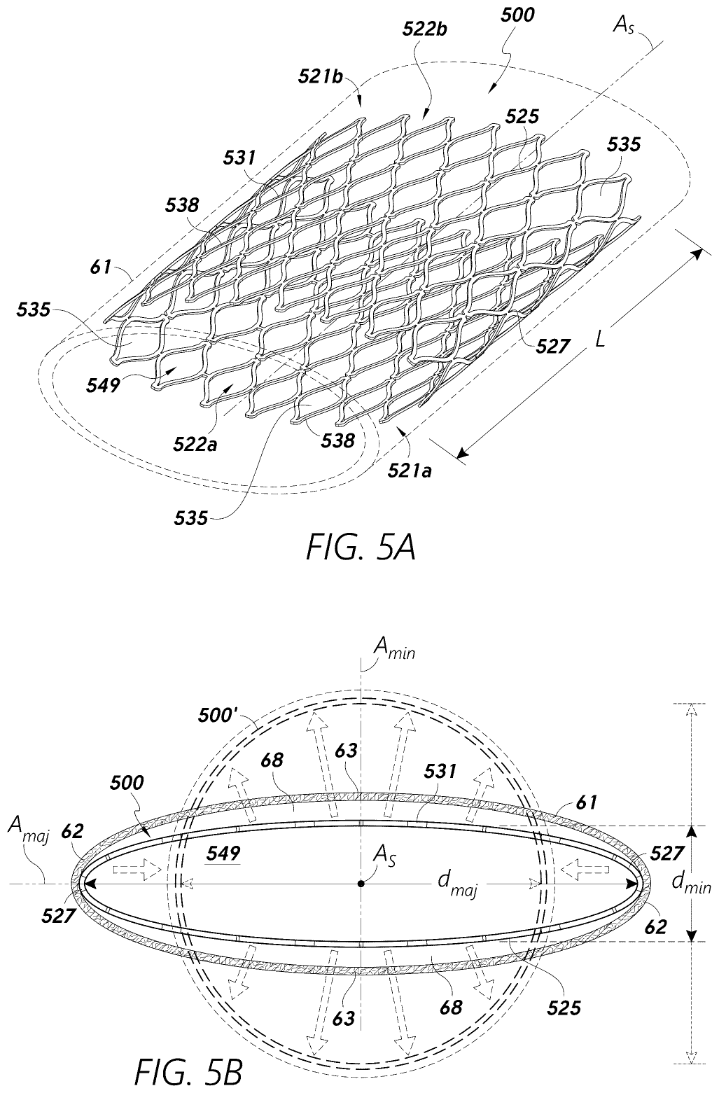

FIGS. 5A and 5B show perspective and axial views, respectively, of a non-circular stent 500 in accordance with one or more examples. Although not shown for clarity in FIGS. 5A and 5B, it should be understood that the stent 500 may comprise one or more hooks, barbs, and/or other attachment features/means adapted to facilitate secure attachment of the stent to the tissue of the target blood vessel wall. Description of aspects of any example tissue-engagement element/feature of the present disclosure may be understood to be implementable in example stents like that shown in FIGS. 5A and 5B. The illustrated stent 500 may represent a non-circular segment of a stent implant having one or more circular portions/segments, as described in detail herein.

The stent 500 may be formed of a tubular frame 531, which may form a wall around an axial channel 549, thereby defining the channel 549. The stent 500 may be an elongate/elongated stent, in that a length L of the stent is greater than a minor-axis diameter dₘᵢₙ and/or major-axis diameter dₘₐⱼ of the stent 500. As described herein, the frame wall 531 of the stent 500 can be considered a single, circumferentially-wrapped wall, or may be considered to comprise multiple walls, or wall segments. For example, with respect to oval stents and other non-circular stents, as illustrated in FIGS. 5A and 5B, such stents may be considered to comprise sidewall segments 525 that run along relatively long sides of the stent that are aligned generally with the orientation of the major axis/dimension Aₘₐⱼ of the stent, as well as end wall segments 527, which may connect the side walls 525 on major-axis ends of the stent 500. The end walls 527 may be outwardly-curved/concave with respect to an axis Aₛ of the stent 500. The sidewalls 525 may bow/deflect outward, either in a resting, unpressurized state, or in conditions of hoop/wall stress on the frame 531. For example, the sidewalls 525 may bow outward such that the sidewalls 525 are concave from the perspective of the axis Aₛ of the stent 500 and convex from the perspective of the exterior of the stent 500.

Certain stent shapes are described herein, including circular, non-circular-, oval-, peanut-, and other-shaped stents. It should be understood that such description of stent shapes refers to a shape of an axial cross-section of a stent, as depicted in the view of FIG. 5B. Although oval- and peanut-shaped stents are described, it should be understood that the principles of the present disclosure may relate to stents having any non-circular shape in at least some configurations thereof (e.g., in a relaxed/biased configuration), and tissue-engagement elements disclosed herein can be configured to facilitate tissue-to-frame couplings of stent segments having non-circular shapes that are other than the illustrated oval- and peanut-shaped stents. Descriptions of stents in a relaxed or biased configuration should be understood to relate to a configuration that a stent naturally assumes in the absence of tension on the stent wall(s) from external forces (e.g., ambient fluid pressure, physical contact forces, etc.). For example, the biased/relaxed shape of the stent may be due to shape memory of the stent and/or frame thereof.

The stent 500 may be considered an oval stent with respect to the shape of the axial cross-section thereof, as shown in FIG. 5B. The term “oval” is used herein according to its broad and ordinary meaning and may be used substantially interchangeably with the term “ellipse” and/or “oblong,” which terms are likewise used according to their broad and ordinary meanings. The term “oval” may be used to refer to any non-circular closed curve having major and minor axes, the major axis being greater than the minor axis. With respect to “oval”-shaped stents disclosed herein, such stents may have relatively flatter minor-axis sidewalls (compared to curved major-axis end walls), wherein the sidewalls may bow radially outward, and/or may be deflected/curved radially inward so as to produce external concavity and internal convexity in such sidewalls (e.g., forming a peanut-shaped stent). Major-axis walls of an oval stent as described herein may be considered wall portions of a stent that are intersected by a major axis of the stent that runs through an axial center of the stent. Minor-axis walls of such oval stents may be considered wall portions that are intersected by a minor axis of the stent that runs through the axial center of the stent. Example stents of the present disclosure may be considered to have an oval shape whether or not the shape thereof is definable by an algebraic curve. Example stents of the present disclosure may be considered oval stents when the wall(s) of the stent in an axial-cross-sectional perspective form(s) a closed or open curve in a plane that is non-circular; one or more segments/areas thereof may resemble the outline of a portion of an egg. Oval stents of the present disclosure may include either one or two axes of symmetry of an ellipse, such as the illustrated major Aₘₐⱼ and minor Aₘᵢₙ axes. The axial cross-section of some examples of oval stents of the present disclosure may resemble the union of two semicircles on opposite sides of a rectangle, providing a shape evoking the likeness of a speed skating rink or an athletics track. In some contexts, the oval stent 500 may be considered a “stadium”-shaped stent, or an elongated oval.

The stent frame 531 comprises stent wall(s) defining an elongated tubular structure having a first axial end 521a with a first opening 522a. The tubular structure may further comprise a second axial end 521b with a second opening 522b, wherein the lumen/channel 549 extends between the first opening 522a and the second opening 522b, traversing the length L of the stent 500. The frame 531 and/or wall(s) thereof may comprise an open-cell structure adapted to be expanded to secure the stent 500 to a blood vessel internal (or external) wall, such as through endothelialization of the frame 531 to the vessel tissue over time as the frame 531 holds the blood vessel wall using certain tissue-engagement features described in detail herein.

The stent 500 may be elastically deformable between a first, non-circular configuration and a second, more-circular configuration 500′ (see dashed-line representation in FIG. 5B), with the stent 500 biased toward the non-circular configuration. In some examples, the stent frame 531 may comprise a shape-memory material, such as Nitinol. Although shown as an oval-shaped stent, the stent 500 may be any non-circular shape in a relaxed state thereof, such as a triangle, peanut, figure-8, star, clover/lobed, and/or kidney shape.

The stent 500 may be configured to be percutaneously delivered to a blood vessel 61 in a compressed delivery configuration. Once within the blood vessel lumen at the target deployment site, the stent 500 may be configured to be radially expanded into direct surface contact with the blood vessel wall (e.g., the inner wall of an aorta segment). In some examples, the stent 500 may be configured to be expanded such that the perimeter of the stent 500 approximates and/or exceeds a perimeter of the blood vessel portion where the stent 500 is implanted, at least immediately prior to deployment/expansion of the stent. Placement of the stent 500 in the blood vessel may cause at least slight stretching in the blood vessel wall, such as due to pressure at the major-axis ends 527 against the blood vessel wall. In some cases, a stent configured to expand to a greater perimeter than the native blood vessel may provide improved traction and/or resistance to migration within the blood vessel. Implanting a stent that has a perimeter approximate to and/or slightly greater than the blood vessel perimeter may increase positive engagement with the blood vessel wall and/or maximize a compliance effect. The stent wall and/or a portion thereof may be configured to be endothelialized to the blood vessel wall.

In the oval configuration shown in FIGS. 5A and 5B, the stent 500 may have a cross-sectional area having a major/long-axis diameter dₘₐⱼ that is substantially larger than the minor/short-axis diameter dₘᵢₙ. For example, the major-axis diameter/dimension dₘₐⱼ may advantageously be at least twice as long as the minor-axis diameter/dimension dₘᵢₙ, or even 3, 4, 5, 6, or 7 times greater. The stent 500 may be configured to increase compliance of a blood vessel though constant or near-constant pressure at one or more points along a perimeter/circumference of the blood vessel that causes a change in the perimeter geometry of the vessel. For example, the blood vessel may be changed and/or moved from a non-circular/less-circular shape to a circular/more-circular shape.

The stent frame wall(s) 531 may be at least partially composed of struts 538 and/or stent openings/cells 535 between the struts 538. The dimensions and/or shape of the stent 500 may vary based on the particular application and/or target implantation anatomy. For example, the stent length L may be selected to extend over all or a portion of an identified non-compliant length of a target blood vessel. The stent major axis dₘₐⱼ and minor axis dₘᵢₙ, when averaged, may be approximately equal to the diameter of the native blood vessel. For example, for a stent configured for deployment in an aorta, the length L may be between 1-30 cm, and in the biased oval/diastolic configuration the major axis dₘₐⱼ may be between 1-4 cm (or larger/smaller depending on the particular anatomy), and the minor axis dₘᵢₙ can be between 20-50 percent of the major axis dₘₐⱼ. However, other sizes and/or shapes are also within the scope of this disclosure.

The configuration of the stent 500 in the oval shape can cause blood vessel wall 61 to assume a more oval shape to match the shape of the stent 500. However, depending on the relative size of the stent 500 to the vessel 61, the blood vessel 61 may not necessarily conform exactly to the circumference and/or shape of the stent 500, and gap(s) 68 may be present and/or form between the frame 531 and the blood vessel wall 61 as the luminal pressure increases and pushes the vessel side walls away from the frame sidewall 525. Due to the presence of the gaps 68, which may form cyclically as the pressure drops (e.g., during diastole) and the stent transitions to the oval shape, such that the walls 525 pull farther away from the blood vessel wall, desirable sealing between the stent and the blood vessel may be impeded. The presence of the gaps 68 can reduce the ability of the stent 500 to reshape the blood vessel, thereby negatively impacting the efficacy of the stent 500 with respect to compliance-enhancement. Furthermore, blood may collect and/or stagnate to some degree in the gaps 68, resulting in increased risk of embolus/thrombus formation.

When implanted in a blood vessel, the patient physiology may respond to the stent 500 as a foreign object. For example, macrophages can accumulate around the stent, and nearby smooth muscle cells can proliferate to cover the stent. Over time, a new endothelial layer can form over the stent, which can inhibit clot formation. In addition to preventing embolus/thrombus formation, endothelialization can enhance the ability of the stent to reshape the target blood vessel by strengthening the physical coupling between the stent and the blood vessel, thereby reducing the presence of gaps forming between the stent and blood vessel wall when the stent walls pull away from the blood vessel wall as the stent reshapes to an oval/non-circular shape. Tissue overgrowth can be promoted through contact between the stent frame and the blood vessel walls. Examples of the present disclosure provide devices that facilitate contact between non-circular stent frames and blood vessel walls using tissue-engagement features associated with the stent frame, thereby increasing the efficacy of the stents with respect to reshaping/compliance-enhancement.

The stent 500 may be biased toward the illustrated oval and/or other non-circular relaxed/diastolic configuration (shown in solid-line in FIG. 5B), and may, when subjected to mechanical forces associated with high luminal pressure, be configured to responsively transform to a more-circular systolic configuration (shown in dashed-line in FIG. 5B) such that the minor axis dₘᵢₙ approaches, and may equal, the major axis dₘₐⱼ. As with any of the examples disclosed herein, the stent 500 can be configured to deflect from the oval shape to a more-circular shape in the presence of threshold blood pressure levels greater than 80 mmHg, such as blood pressure levels greater than 90 mmHg (e.g., between 90-120 mmHg).

For examples in which the stent frame 531 is not covered by a fluid-tight covering, the cells 535 of the frame 531 can provide openings in the frame 531 that allow blood in the blood vessel 61 to transfer pressure through the frame 531, to thereby load the inner diameter/surface of the blood vessel with a force resulting from increases in the luminal blood pressure as the heart beats. For example, luminal pressure forces against the blood vessel wall increase the hoop stress on the blood vessel, which may force the blood vessel, and with it the stent 500, to assume a more-circular shape. The resulting hoop stress from luminal pressure increase can exert radially-outward force along the blood vessel's inner circumference, such stresses/forces being tensile in nature, which can tend to cause the blood vessel to increase in diameter. The blood pressure force on the blood vessel wall and resulting inward deflection of the blood vessel wall (due to outward deflection of the vessel wall portions in the area of the minor axis Aₘᵢₙ) at the major-axis-ends 527 of the stent 500 may cause inward deflection of the ends 527 of the stent 500 to form a desired geometric change to a more-circular shape 500′ (shown in dashed line) of the stent and the blood vessel 61. The transition of the stent from oval to the more-circular stent shape 500′ (shown in dashed-line in FIG. 5B) causes energy to be stored in the stent frame 531 (e.g., in the elasticity and/or shape memory thereof), such that energy is returned to the blood vessel walls, and therefore to the blood circulation within the blood vessel segment, when the frame transitions back to the oval shape 500 as pressure decreases.

For examples in which the stent frame 531 is covered internally and/or externally by a fluid-tight covering (not shown in FIGS. 5A and 5B for visual clarity), the openings of the cells 535 of the frame 531 may be closed to pass-through fluid and prevent blood in the blood vessel 61 and within the channel 549 of the stent 500 from transferring pressure through the frame 531. Therefore, intraluminal pressure within the flow channel 549 of the stent 500 loads against the frame 531 (rather than directly against the blood vessel walls 63) to provoke reshaping thereof. The blood-pressure-induced force against the covering and/or stent frame 531 can increase the hoop stress on the frame 531 and/or covering, which may force the frame 531, and with it the blood vessel 61, to assume a more-circular shape. Unlike with bare-frame stents that do not include fluid-tight coverings, where a covering is present, the endoluminal pressure forces in the channel 549 of the covered stent 500 are distributed over/against the covering and/or frame 531 to directly reshape the stent 500, rather than indirectly reshaping the stent 500 through force on a finite number of end contact/pressure points of the frame 531. As the pressure in the channel 549 increases (e.g., in connection with the systolic phase of the cardiac cycle), the plastically-deformable nature of the stent frame 531 allows for the sidewalls 525 of the frame 531 to be pushed outward along with the shortening of the stent 500 in the major axis dimension Aₘₐⱼ. When the sidewalls 525 are deflected outward, the channel 549 assumes a more circular cross-sectional shape.

The natural cross-sectional shape of the aorta (and other blood vessels) may generally be circular. With the oval stent 500 pushing outward to the oval configuration, the wall portions 63 may be pulled/drawn at least partially towards an axial center of the blood vessel 61 and/or towards each other in a manner as to cause the blood vessel 61 to form a non-circular shape, such as the oval shape shown in FIG. 5B. However, where the blood vessel 61 does not necessarily conform exactly to the circumference and/or shape of the oval stent 500, the gap(s) 68 may be present and/or form between the frame 531 and the blood vessel wall 63 as the luminal pressure increases and pushes the vessel side walls 63 away from the frame sidewall 525.

With respect to covered stent examples, the biasing/shape-memory of the stent frame 531 may cause the sidewalls 525 to naturally pull towards the center of the frame 531, thereby potentially pulling away from the vessel sidewalls 63 to form gap(s) 68 between the stent walls 525 and the vessel walls 63 if insufficient tissue coupling between the stent 500 and the vessel 61 has occurred. Therefore, as with bare-frame stent examples, it may be desirable to promote tissue-coupling/engagement between non-circular stents and a blood vessel, which can promote tissue ingrowth and/or increase the reshaping effect of the stent.

The stent 500 may be deployable within a blood vessel lumen, as described and illustrated herein. However, it should be understood that example stent devices of the present disclosure may alternatively or additionally be deployable in a position around an outer surface of a target blood vessel, in which case tissue-engagement features of the stent may project radially-inwardly to engage with the blood vessel wall.

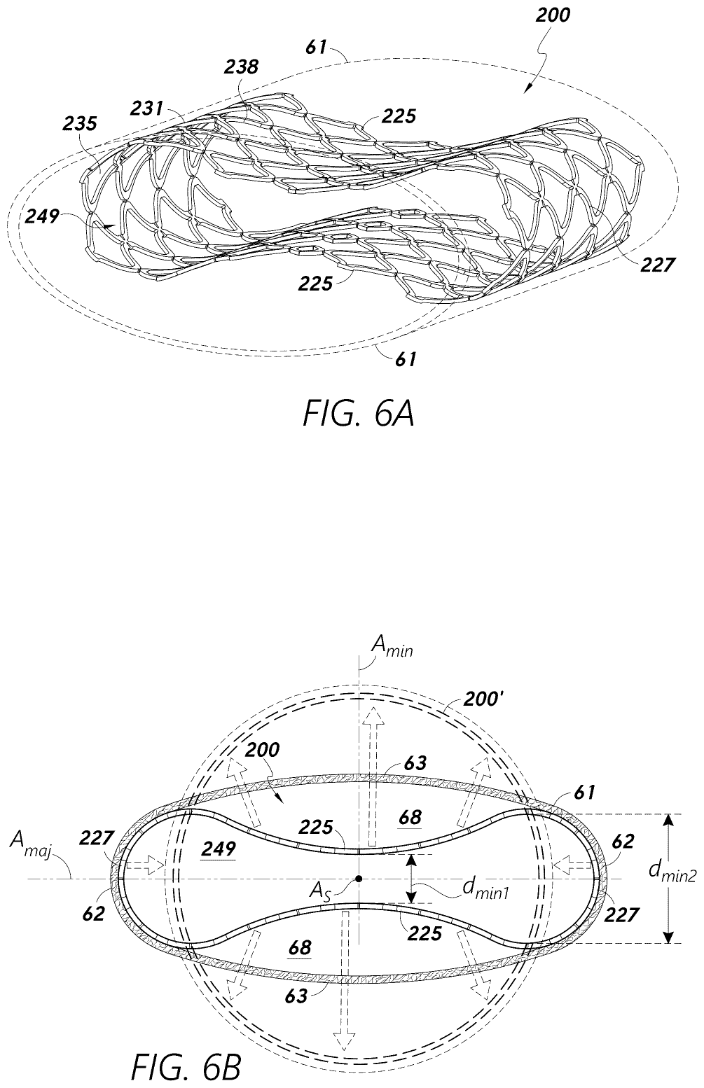

FIGS. 6A and 6B show perspective and axial views, respectively, of a non-circular stent, or stent segment, 200 having inwardly-deflected sidewalls 225 in accordance with one or more examples. The stent 200 may represent an example implementation of any of the non-circular/oval stents/stent-segments disclosed herein, or portion(s) thereof. The shape of the stent 200 deviates from the stent 500 shown in FIGS. 5A and 5B only in that the minor-axis (e.g., relatively flat and/or long) sidewalls 225 deflect to a greater degree towards the center/axis Aₛ of the stent 200 in a relaxed state. The resulting shape may resemble that of an hourglass and/or peanut shape with respect to the axial cross-section shown in FIG. 6B. The stent 200 may have a major axis diameter/dimension that is greater than a minor axis diameter/dimension of the peanut cross-section.

In some examples, the stent 200 may be biased to a shape having a minor axis dimension dₘᵢₙ that is non-constant along the major axis Aₘₐⱼ dimension, which forms an externally-concave/internally-convex surface/form with respect to the minor axis sidewalls 225. For example, the minor axis sidewalls 225 may have a diameter dimension dₘᵢₙ₁ at a center thereof (with respect to the major axis dimension Aₘₐⱼ) that is less than the diameter/dimension dₘᵢₙ₂ at/towards the end portions of the minor-axis sidewalls 225. Non-circular stents of the present disclosure that have externally-concave/internally-convex minor-axis sidewalls as shown in FIGS. 6A and 6B are referred to herein as peanut-shaped stents; such peanut shape can be considered a variation of an oval, or oval-shaped, stent as described herein. The inwardly-deflected walls 225 can allow for a transition reshaping between peanut-shaped, to outwardly-bowed oval shape, and ultimately to circular/more-circular shape, shows as the dashed circular stent shape 200′.

Compared to the oval stent 500 of FIGS. 5A and 5B, the peanut-shaped stent 200 may have a tendency to form even greater gaps 68 between the minor-axis sidewalls 225 and the blood vessel walls 61 due to the inward deflection of the walls 225 away from the blood vessel wall. That is, the tendency of the stent sidewalls 225 to pull away from the blood vessel sidewalls 63 may be relatively high, and therefore, without tissue coupling between the vessel walls 63 and the stent walls 225, the reshaping ability/function of the stent 200 may be at least partially impaired/impeded. Furthermore, where the gaps 68 are present between the stent walls 225 and the vessel walls 63, blood may collect and stagnate in such spaces, presenting embolism risk and/or impeding outward deflection of the stent walls 225 to circularize during the systolic phase of the cardiac cycle. Therefore, facilitating tissue overgrowth of the stent walls 225 may be of particular significance with respect to peanut-shaped stents.

For bare-frame peanut-shaped stent configurations, the gaps 68 between the stent walls 225 and the vessel walls 63 may form as the luminal pressure increases and pushes the vessel side walls 63 away from the frame sidewalls 225, which in-turn forces the end walls of the vessel 62 and the end walls 227 inward to cause the stent sidewalls 225 to outwardly deflect to the more-circular shape 200′ shown in dashed-line representation in FIG. 6B. For covered peanut-shaped stent configurations, increases in luminal pressure may directly push against the sidewalls 225 (and/or covering) to cause outward deflection thereof. For covered and non-covered implementations, as pressure decreases, the shape memory of the frame 231 causes the sidewalls 225 to pull back away from the vessel walls 63 to form the gaps 68.

In order to promote tissue ingrowth between sidewalls of non-circular (e.g., oval, peanut-shaped) stents of the present disclosure and blood vessel walls, certain tissue-engagement, anchors, spikes, barbs, hooks, or the like can be implemented to couple the minor-axis walls of a non-circular-biased stent to a target blood vessel wall to conform the blood vessel walls more closely to the shape and configuration of the stent frame walls.

When a minor-axis wall of a non-circular stent of the present disclosure is coupled to a blood vessel wall via one or more anchors or other tissue-engagement features, the stent may be able to pull the blood vessel wall inward to more effectively reshape the blood vessel to improve diastolic blood flow. Furthermore, such coupling can facilitate endothelialization of the blood vessel wall over the stent frame and/or covering associated therewith. Generally, the deployment of the stent in the target blood vessel can cause injury to the vascular wall and endothelium, which comprises a layer of endothelial cells lining the vascular wall that promotes homeostasis. The injury to the blood vessel wall can result in inflammation, repair, and the development of neointimal hyperplasia. The ability of the endothelium to repair itself can depend on the migration of surrounding mature endothelial cells, and the attraction and adhesion of circulating endothelial progenitor cells (EPCs) to the area where the stent is deployed, forming overgrowth of endothelial-like cells. In some examples, stent frames and/or coverings can be implemented with drug-eluting features/coatings that at least partially interrupt the natural tissue overgrowth process. Alternatively, stents can be implemented with treatments that accelerate the reendothelialization of the damaged arterial segment following stent deployment. Such tissue-growth promotion can reduce risks of neointimal hyperplasia and stent thrombosis. In order to promote the desired coupling between stent walls and blood vessel walls to facilitate blood vessel reshaping as described in detail herein, stent frames and/or coverings of stents of the present disclosure can be treated with agents that augment the generation and/or accumulation of endothelial cells or EPCs in the area of the deployed stent. In some implementations, the stent frame and/or covering can be seeded with endothelial cells or EPCs. For example, a stent can be coated with anti-CD34 antibodies to attract endothelial/EPC cells to the stent. The stent-reshaping/circularizing structures disclosed herein can be designed to hold the associated stent in a relatively circular shape for a period of time that correlates with the expected tissue overgrowth period based on the tissue-growth-promoting/inhibiting features of the stent implant.

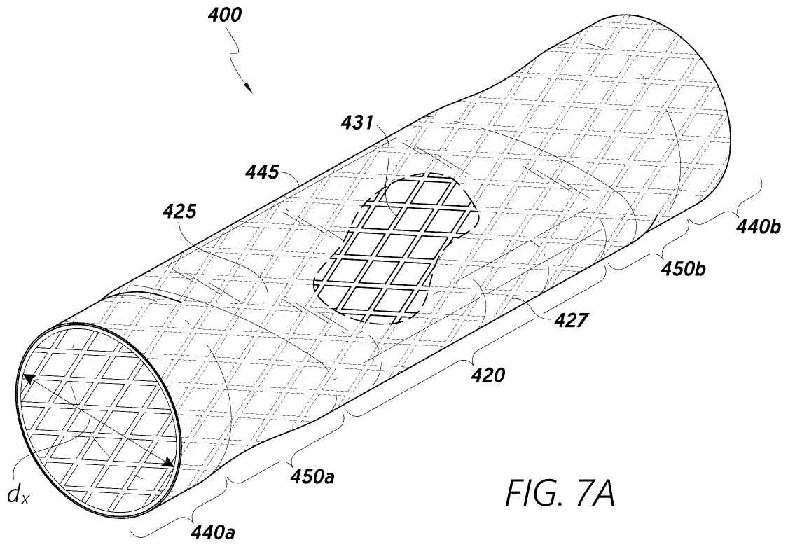

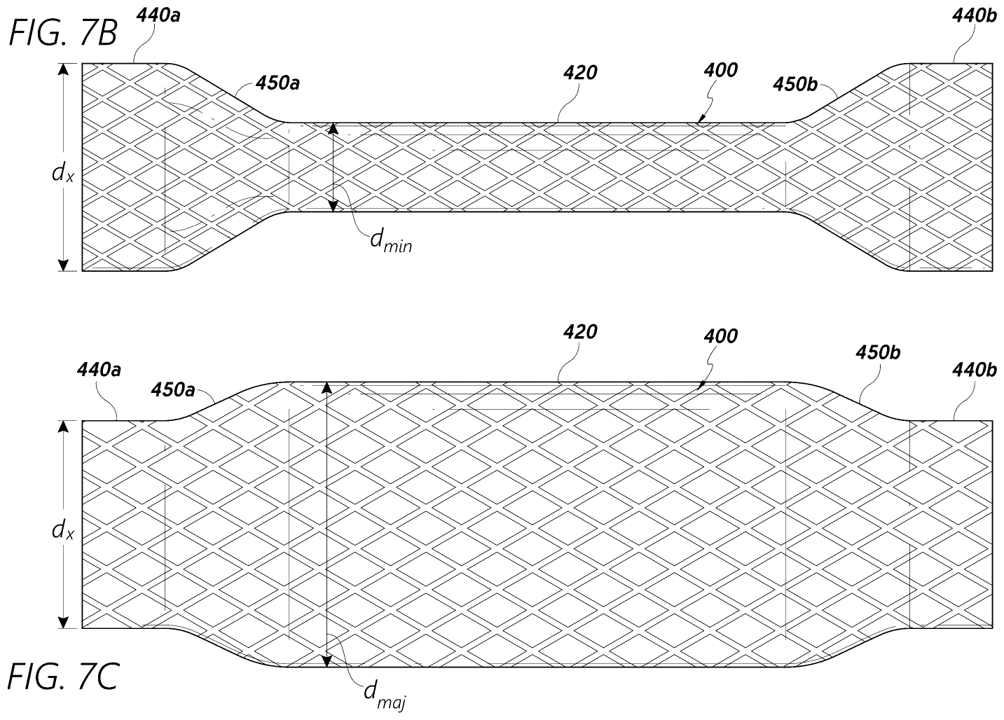

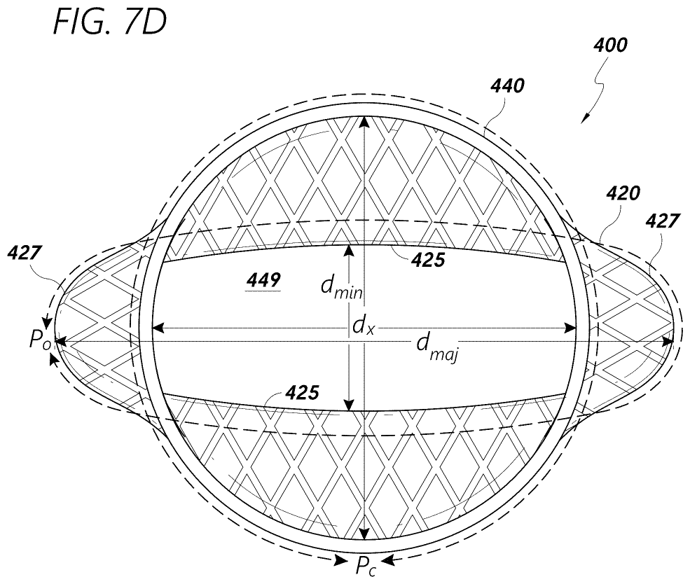

FIGS. 7A-7D show perspective, side, and axial views, respectively, of a stent 400 having circular end portions 440 and a non-circular (e.g., oval) medial portion 420 in accordance with one or more examples. Stent devices having circular axial end portions that provide direct circular contact with the blood vessel at the ends of the stent can particularly benefit from the implementation of tissue-engagement features as disclosed herein with respect to non-circular medial portion(s) thereof. For example, blood collection/stagnation outside such medial stent portions can be reduced and/or eliminated through effective implementation of tissue coupling between the stent frame and the blood vessel using any of the anchors, spikes, barbs, etc. disclosed herein.

As described in detail throughout the present disclosure, oval-shaped and other non-circular stents can be utilized to improve compliance characteristics of a target blood vessel or blood vessel segment. Due to various hemodynamic considerations, as referenced above, it may be desirable to implement such stents in a manner such that a hemostasis seal is present/facilitated on axial ends of the stent. Furthermore, due to the natural circular/cylindrical shape of the blood vessel, oval stents of some examples may not fit as securely within a target blood vessel as compared to traditional circular/cylindrical stents of the same size. For example, while a circular/cylindrical stent may generally distribute contact with the blood vessel wall evenly around the circumference thereof, such that contact between the stent and the blood vessel is not concentrated in specific contact points around the circumference of the stent, oval stents may have a tendency to contact the blood vessel primarily at the major axis ends of the stents oval form, as described in detail above. Furthermore, due to the dynamic reshaping of certain oval stents as described herein, minor axis sidewalls of the oval stent may become cyclically spaced/separated from the adjacent blood vessel wall, further compromising the secure axial fixation of the stent within the blood vessel and/or the hemostatic seal/barrier between the stent and the vessel wall. Therefore, oval stents in accordance with aspects of the present disclosure can benefit from utilization with certain vessel-anchoring features to further facilitate/improve the axial fixation of such stent devices in the target blood vessel segment and/or promote hemostasis between the stent and the blood vessel wall and/or prevent the collection of blood between the stent and the blood vessel wall.

In some implementations, stent device of the present disclosure include stent/frame portions that have a circular/cylindrical relaxed shape/form, wherein such stent portions can be integrated with oval portion(s) of the stent in some manner, such that the circular portion(s) of the stent serve to securely hold the stent in place in the blood vessel, whereas the oval portion(s) of the stent can function to increase blood flow (e.g., diastolic flow) through the stent as the oval stent portion transitions between oval and circular/cylindrical configurations in response to changing pressure conditions, as described in detail herein.

The stent 400 includes circular stent portions 440a, 440b, which are associated with respective axial ends of the stent 400. Although circular portions 440 are shown on both axial ends of the stent 400, it should be understood that circular-oval stent devices of the present disclosure may include only a single circular portion on one end of the device in some cases. The circular stent portions 440 can have any suitable or desirable axial length. In some implementations, the circular portions 440 have a length (in the dimension L) that is less than the oval portion 420. For example, the oval portion 420 may have a length that is at least twice as long as either of the circular portions 440, or twice as long as the combined lengths of the circular portions 440.

The circular portions 440 may be formed of portions of a stent frame 431, which may be integrally formed with the frame portion forming the oval portion 420 of the stent 400. For example, the frame 431 may transition between the circular shape of the circular portions 440 and the oval shape of the oval portion 420. The frame 431 may transition in a relatively smooth/gradual manner from the shape of the oval segment 420 to the circular shape of the circular segments 440 in transition portions/segments 450 of the stent 400. That is, with respect to the flat/long sidewalls 425 that run in the major axis dimension of the oval portion 420, the diameter of the frame 431 may transition from a narrow minor-axis diameter dₘᵢₙ to the circular diameter dₓ of the circular portions 440 moving axially from the oval portion 420 towards the circular end portions 440. Furthermore, with respect to the curved/short end walls 427 of the oval portion 420 that curve around the major axis ends of the oval form thereof, the diameter dₘₐⱼ associated therewith may transition from the relatively long dimension dₘₐⱼ to the relatively shorter circular diameter dₓ moving from the oval portion 420 through the transition portions 450 to the circular portions 440. In some examples, as shown in FIGS. 7A-12D, the diameter dx of the circular portions 440 may be less than the major diameter dₘₐⱼ of the oval portion 420, but greater than the minor diameter dₘᵢₙ of the oval portion 420.

As shown in FIG. 7A, the stent 400 may include a covering 445, which is illustrated on an outer surface of the stent 400, although it should be understood that such covering 445 may be disposed within the frame 431 on an inner side/diameter thereof and/or within and without the frame 431, as with other examples of the present disclosure. For clarity, the covering 445 is shown only in FIG. 7A. While a stent frame 431 and covering 445 are shown in FIG. 7A, wherein the covering provides a fluid-tight surface against which pressurized blood within the channel 449 of the stent 400 can press to reshape the oval portion 420 of the stent 400, it should be understood that in some examples, the stent 400 comprises a planar/sheet form that is fluid tight in one or more portions thereof, wherein such planar/sheet form provides the structure/frame of the stent 400 and substitutes for the illustrated strut-based frame 431 and covering 445.

The stent 400, which may be a covered or bare-frame stent, is formed with the central/medial oval-shaped segment 420, which assumes and oval or other non-circular shape at least in a free/relaxed state thereof, with both ends of the stent having circularly-shaped forms so as to fully-engage with the surrounding vascular wall. In some implementations, the end portions 440 can be relatively oversized to conform with and press against the blood vessel walls, which can improve hemostasis, thereby restricting blood flow to solely through the stent's lumen 449. The oversizing of the circular portions 440 relative to the native blood vessel can further help the stent 400 resist migration in either axial direction.

The stent 400 and/or frame 431 thereof may have the same perimeter length P_{c} in the circular portions 440 as in the oval portion 420 (perimeter Pₒ shown in FIG. 7D). In some implementations, the perimeter Pₒ of the circular portions 440 is greater than the perimeter P_{c} of the non-circular portion 420. The transition segment/portions 450 may likewise have the same perimeter as the end 440 and oval 420 segments. The perimeter P_{c} of the circular portions 440 may be selected to match the circumference of the target blood vessel segment. For example, the perimeter P_{c} may be within 10% of the length of the circumference of the target blood vessel in the implantation area. The circumference/perimeter P_{c} of the stent may be slightly larger than that of the target blood vessel prior to deployment of the stent 400, to thereby promote secure attachment of the stent 400 to the blood vessel.

When the stent 400 is implemented with a covering 445, the circular end-portions 440 can serve as funnels that restrict blood flow to flowing through the channel of the medial portion 420, and not flowing around the outside of the medial portion 420. However, as the circular end portions 440 may not be configured to change in shape throughout the cardiac cycle as pressure changes, such portions may not contribute to the stent's compliance-enhancing, vessel-reshaping effect. Moreover, the circular shape of the end portions 440 may cause the end portions to serve as stationary harnessing portions that may limit the movement of the central/medial 420 portion between systolic and diastolic phases. Furthermore, the relatively abrupt transition from the enlarged circular shape of the end portions 440 to the narrower profile of the non-circular medial portion 420 shaped portion may result in certain local flow disturbances, and/or pose manufacturing complications associated with increased deformations/strains. Therefore, it may be desirable to implement the end portions 440 as non-circular portions in some implementations.

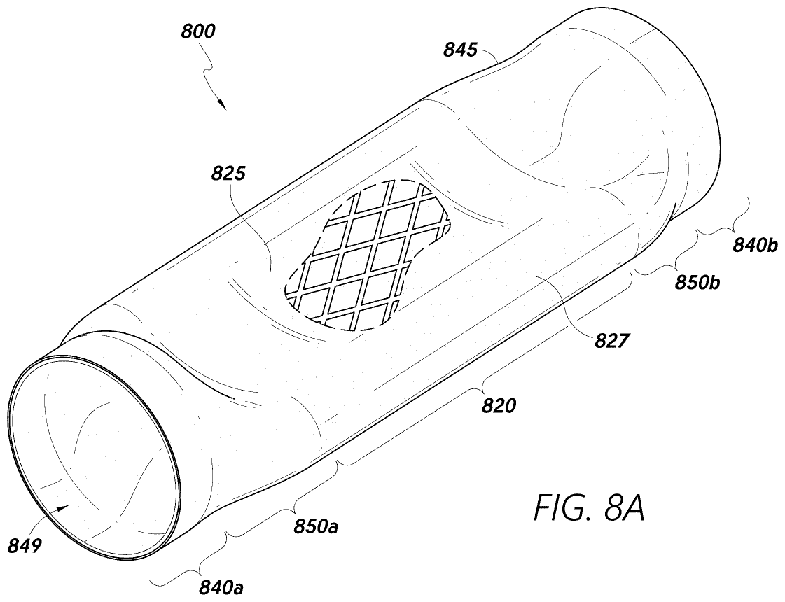

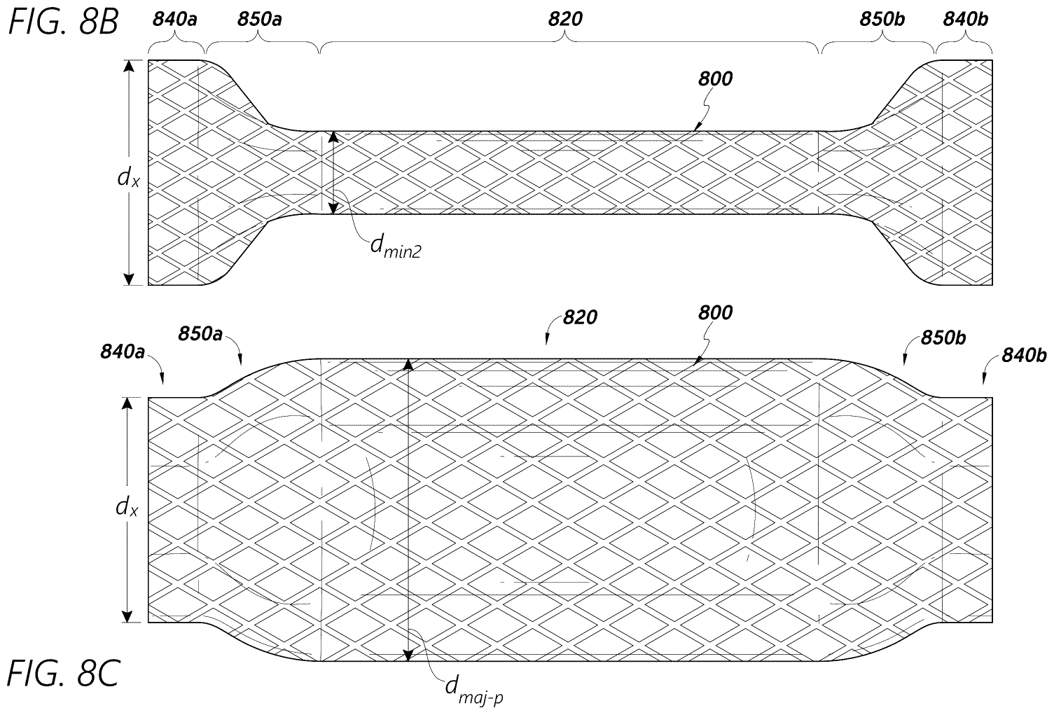

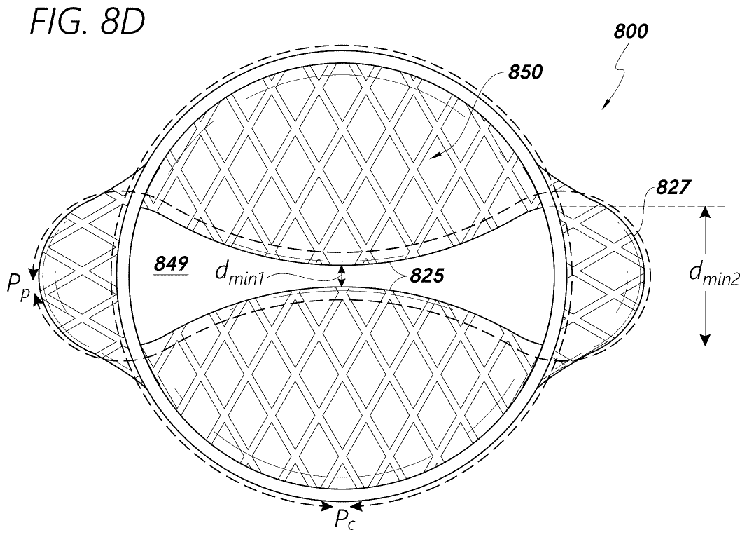

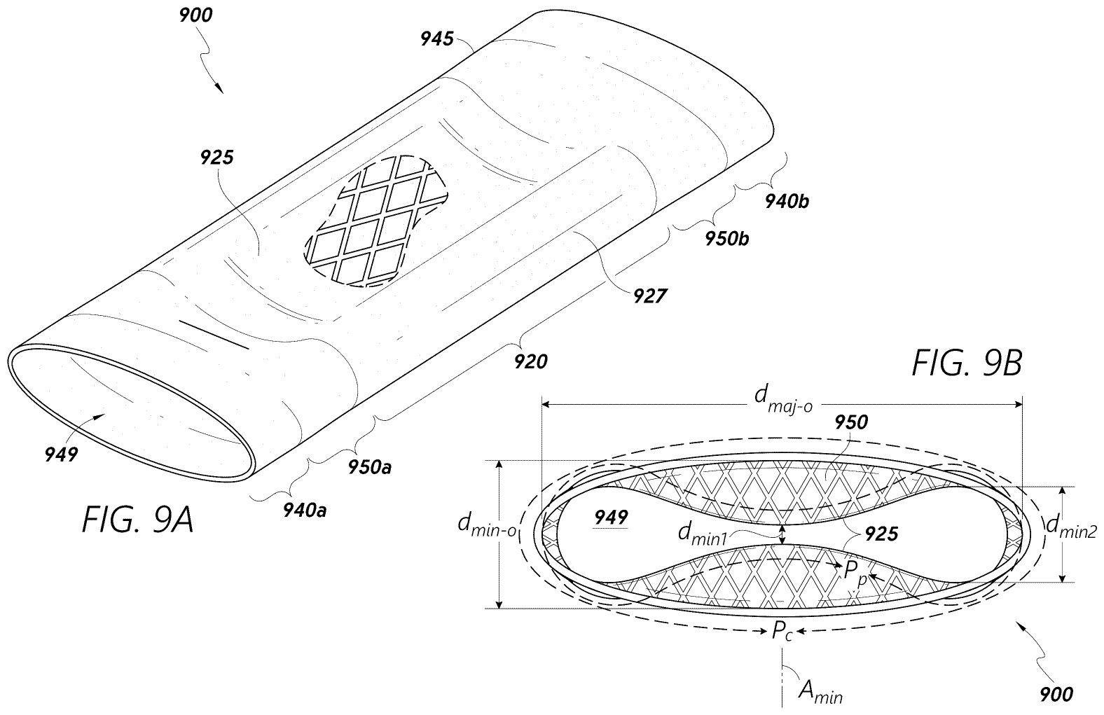

As described above, in order to provide the desired vessel-reshaping functionality, stent devices having non-circular segments thereof may benefit from the implementation of coverings, which may increase the reshaping force on the stent frame by channeling blood that passes through the blood vessel segment spanned by the stent implant device through the inner channel of the stent, rather than in the space outside of the stent frame. Therefore, the embodiment of FIGS. 7A-7D, and other stent implant devices disclosed herein (e.g., the device 800 of FIGS. 8A-8D, the device 900 of FIGS. 9A and 9B, and other stent implant devices disclosed herein), can advantageously be implemented with coverings to improve the functionality thereof. Alternatively, examples of the present disclosure may be implemented without coverings, wherein the blood vessel wall itself may serve as a covering over the outer diameter of the stent frame to increase the effect of luminal pressures on the stent frame. For example, tissue-engagement features/elements of examples of the present disclosure may be implemented that cause the vessel wall to be physically coupled to the outer diameter of the stent frame in a manner such that pressure forces against the blood vessel wall pull on the stent frame and can cause a reshaping thereof, and further, reshaping of the stent can pull on the blood vessel walls as well. In such implementations, the blood vessel wall itself may be used as the covering of the stent.

Vessel-to-stent coupling can be of particular benefit for stents including peanut-shaped stent segments, due to the desire to effect relatively greater reshaping/force on the peanut-shaped stent frame to transition to a circular shape during systole or other high-pressure condition. FIGS. 8A-8D show perspective, side, and axial views, respectively, of a stent 800 having circular end portions 840 and a non-circular (e.g., peanut-shaped) medial portion 820 in accordance with one or more examples.

The stent 800 may have any of the features disclosed above in connection with the stent 400 of FIGS. 7A-7D. However, with respect to the medial portion 820 of the stent 800, such portion may have a cross-sectional axial shape resembling the peanut-type shape disclosed in FIGS. 6A and 6B. Similarly to the stent 400 of FIGS. 7A-7D, the stent 800 can include axial circular ends 840 and transition portions/segments 850 that transition the shape of the stent 800 between the circular ends 840 and the peanut-shaped medial portion 820.

The medial portion 820 may have any suitable or desirable length, and may comprise inwardly-deflected minor axis walls 825, which are connected at major axis ends 827 as with other examples disclosed herein. Although a particular peanut shape is shown for the medial section 820, as illustrated clearly in the axial view of FIG. 8D, it should be understood that the medial segment/portion 820 may have any suitable or desirable degree of deflection/curvature, or other features defining an at least partially peanut-shaped conduit. For example, the medial portion 820 may have any suitable minimum diameter dₘᵢₙ₁ (see FIG. 8D) and/or any suitable or desirable maximum minor-axis dimension dₘᵢₙ₂. Furthermore, the curvature and/or angle(s) of deflection of the walls 825/827 of the peanut-shaped medial portion 820 may have any suitable or desirable parameters. As shown in FIG. 8A, the stent 800, as with other examples disclosed herein, may or may not comprise a covering 845, which may be disposed on an outer surface and/or inner surface of a frame/skeleton structure of the stent 800. The stent frame 800 can comprise a solid-surface form, rather than a structure formed of struts and open cells as illustrated and described in detail herein.

From the top view of FIG. 8C, it can be seen that in some examples, the peanut-shaped medial portion 820 has a major diameter dₘₐⱼ₋ₚ that may be greater than a diameter dx of the circular portion 840. Conversely, the minor axis dimension dₘᵢₙ₂, as shown in the side view of FIG. 8B, may have a value less than the diameter dₓ of the circular portions 840. As with other similar examples disclosed herein, the circular end portions 840 may be configured to provide a desirable (e.g., hemostatic) seal between the axial end(s) of the stent 800 and the surrounding blood vessel wall, whereas the differently-shaped medial portion 820 may be shaped to cause radial elongation of the blood vessel in which it is implanted, to thereby transition the blood vessel between oval-shaped and more-circular-shaped in the presence of relatively high luminal pressure within the channel 849 of the device 800. That is, in high pressure conditions, the stent 800 may assume a substantially-continuously circular/cylindrical shape throughout the axial length thereof.

In some examples, the perimeter/circumference P_{c} of the circular portions 840 may be substantially equal to the perimeter/circumference Pₚ of the peanut-shaped portion 820 and/or at least a portion thereof. Therefore, when the peanut-shaped portion 820 transitions to a more-circular shape, the cross-sectional shape thereof may resemble that of the circular ends 840.

In some implementations, the present disclosure relates to variations of stent designs having differently-shaped end and medial portions/segments. For example, aspects of the present disclosure can provide stents having end portions that are not circular, but rather some non-circular shape that is different than the non-circular shape (e.g., peanut shape) of the medial portion of the stent. In such examples, one or both ends of a compliance-enhancing stent can be designed, with respect to a relaxed shape/configuration thereof, as compliant oval-shaped portions, which may advantageously retain proper sealing against the blood vessel, while also contributing to the stent's blood-vessel-reshaping functioning during transitions between low (e.g., diastole) and high (e.g., systole) pressure phases/conditions. Such stent end shaped can further form more gradual transitions in shape that reduce the likelihood of flow disturbances through the stent. FIGS. 9A and 9B show a stent 900 having oval end portions 940 with radially-outwardly-deflecting/bowing minor-axis sidewalls and an oval medial portion 920 with radially-inwardly-deflecting/concave minor-axis sidewalls (e.g., peanut shape), in accordance with one or more examples. Although the device 900 is described below as a stent, it should be understood that, as with any other device disclosed herein, the device 900 may be implemented and/or implanted as a graft that replaces a resected blood vessel segment or other gap between blood vessel segments. For example, the end portions 940 of the devices 900 may be sutured or otherwise attached to open ends or openings of/in blood vessel segment(s), such that at least a portion of the length of the medial portion 920 of the device 900 spans a distance between the blood vessel segments and/or is not entirely endovascular when implanted.

The stent shape of the stent 900 of FIGS. 9A and 9B may have certain advantages over other stent designs with a peanut-shaped medial portion and including non-compliant circular end portions. For example, while for some designs with circular end portions, such end portions may not be configured to change in shape throughout the cardiac cycle, and thus may not contribute to the stent's compliance-enhancing vessel-reshaping functionality, the oval end portions 940 of the stent 900 may be configured to change in shape in a cyclical manner, thereby contributing to compliance enhancement. Furthermore, while circle-shaped end portions may limit the transitional movement of a stent's central/medial portion, the oval-shaped end portions 940 may be less limiting with respect to the transitional movement of the medial, peanut-shaped portion 920 between the relaxed peanut shape thereof and a more-circular configuration. In addition, while some designs with circular end portions may present relatively abrupt transitions from the circular shaped ends to the narrower (e.g., significantly narrower) peanut-shape of the medial portion of the stent, which can result in flow disturbances, and may present manufacturing complication associated with significant deformations/strains, the transition between the oval shape of the end portions 940 and the peanut shape of the medial portion 920 may have less of an effect on the flow and structural design strains/deformations than some circle-end stents.

The stent design of FIGS. 9A and 9B, when implanted in a blood vessel, may be prone to the collection of blood in the medial portion 920 in the space formed by the inward deflection of the minor-axis walls 925. Therefore, tissue-engagement features as disclosed herein may advantageously be associated with the minor-axis walls 925 to promote blood vessel coupling in a manner as to expel and block blood from the space between the stent frame and the blood vessel. Furthermore, advantageously, the oval shape of the end portions 940 can be configured/dimensioned to retain contact along its entire circumference with the blood vessel (as with circular end portions of examples disclosed herein), thus preserving proper sealing against the artery wall and channeling the flow into the stent's lumen through the medial portion 92. Tissue-engagement features/elements of the present disclosure can facilitate such sealing/coupling between the oval ends 940 and the blood vessel.

As described above, various solutions of the present disclosure using stents having non-circular segments can be implemented for treating hypertensive patients, such as heart failure patients, by improving aortic compliance through blood vessel reshaping. Such stent devices, as described above, can include circular end segments, which may facilitate sealing of the stent implant between the stent and the outer blood vessel wall. For example, stent implants in accordance with aspects of the present disclosure can be configured as a circle-oval/peanut-circle implants, examples of which are shown in FIGS. 7A-7D and 8A-8D, or oval-peanut-oval shaped stents, as shown in FIGS. 9A and 9B. With respect implementations comprising fluid-tight coverings within and/or without the stent frame, such coverings can aid in the reshaping effectiveness and/or blood-flow channeling of the implant.

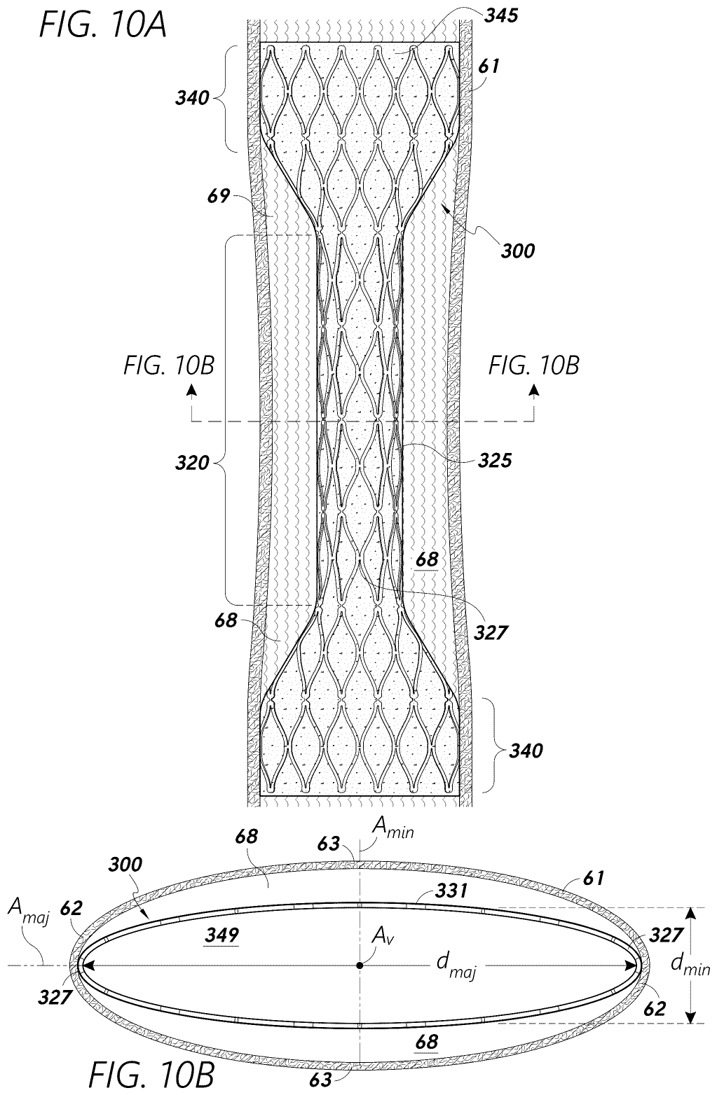

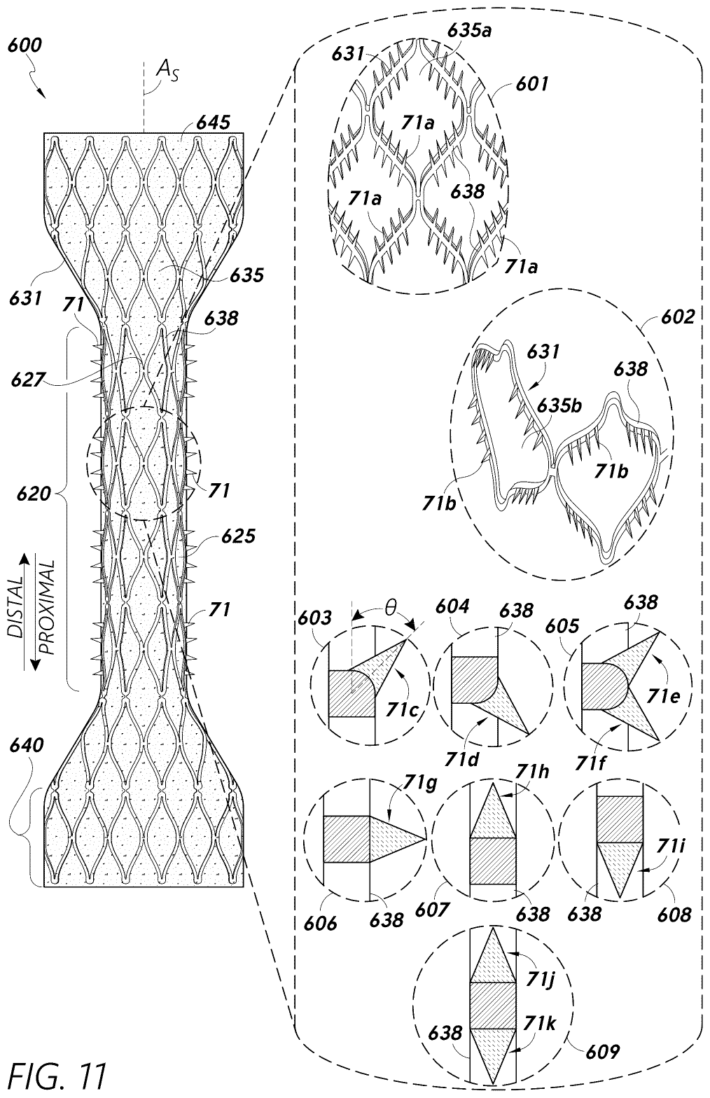

As non-circular stent implants having circular sealing end portions may generally include non-circular medial portion(s)/segment(s) that have a minor axis that is less/shorter than the minor axis of the end segments of the implant, certain volume(s) of blood can become trapped between the stent implant's outer (and/or inner) cover and the blood vessel wall outside of the implant in the blood vessel segment spanned by the medial segment of the stent implant. For example, blood may become trapped between the stent's cover along its non-circular/oval portion between the end segments of the stent and the blood vessel's inner wall. FIGS. 10A and 10B show side and axial views, respectively, of a stent implant device 300 implanted within a blood vessel 61 with trapped blood 69 collected around the stent implant 300 in accordance with one or more examples.

Stagnated blood present in the gaps 68 between the non-circular stent segment 320 and the blood vessel wall 61 can present certain issues. For example, stagnated blood can pose a risk of thrombus formation in the gap area(s) 68, wherein such thrombi formed on the stent implant can compromise the re-shaping functionality thereof. Furthermore, thrombus formation can result in myocardial infarction and/or stroke if dislodged from a position within the gap areas 68 and passing into the bloodstream. Therefore, it may be desirable to implement blood-vessel-reshaping stent implant devices of the present disclosure in a manner as to reduce the presence/occurrence of trapped blood 69 between covered non-circular stent segments and the surrounding vascular anatomy 61, thereby reducing the risk of thrombus and/or otherwise improving the efficacy/functionality of the implant device. Over time, stagnated blood 69 in the gap areas 68 can become solidified and/or tissue-like in a manner that effectively thickens the blood vessel wall 61. Stagnated blood in the gap areas 68 can further defeat the blood-vessel-reshaping effect of the stent due to the generally non-compressible blood 69 occupying the space 68 around the stent frame 331 creating obstruction with regard to the ability of the stent frame to become circularized as pressure levels increase. For example, as the blood vessel segment 61 may have compromised compliance characteristics, the ability of the blood vessel to stretch to accommodate both the expanded circularization of the stent frame 320 and the blood 69 trapped outside of the stent frame may be reduced, and therefore the volume change produced through stent reshaping during cardiac cycling may be limited/reduced, along with the compliance effect thereof.

The stent frame 331 may be inclined to experience tissue in-growth in one or more areas thereof. For example, the major-axis end walls 327 may be relatively stationary relative to the blood vessel walls throughout the cardiac cycle due to the relatively constant contact between the stent side walls 327 and the blood vessel sidewalls 62 between oval and circular shapes of the stent 300. Conversely, in the absence of sufficient tissue growth coupling the stent sidewalls 325 to the vessel sidewalls 63, the sidewalls 325 of the frame 331 and the sidewalls 33 of the blood vessel 61 may tend to come into contact with one another and separate in a cyclical manner as the blood pressure forces the blood vessel sidewalls 63 outward and away from the stent sidewalls 325 during portion(s) of the high-pressure phase/stage of the cardiac cycle. Therefore, tissue ingrowth may occur primarily between the blood vessel end walls 62 and the stent end walls 327 in situations in which tissue ingrowth occurs.

### Stent-Tissue-Coupling Using Anchors

Implementations of the present disclosure can include certain tissue-engagement features, such as spikes, barbs, hooks, screws, anchors, and/or other structures or projections configured to couple a stent implant and/or frame associated with a non-circular segment of the stent implant to the blood vessel wall in a sufficiently close coupling to prevent or reduce the presence of gap space(s) between the stent and the blood vessel wall, thereby reducing or eliminating the available space for trapped/stagnated blood to collect outside of the stent frame.

Examples of the present disclosure can comprise stent implants including certain tissue-engagement features configured to couple of the stent frame to the blood vessel wall of the blood vessel in which the implant is deployed. As referenced above, such coupling between the stent frame and the blood vessel wall can reduce the collection of blood between the stent frame and the blood vessel wall that can otherwise present risks to the patient's health and/or reduce the reshaping ability of the stent implant. Implementation of tissue-engagement elements/features on stent frames can help reduce the collection of blood outside of a stent barrel. In addition, tissue-engagement features of examples of the present disclosure can increase the blood vessel-reshaping effectiveness of a stent implant device by facilitating both pushing and pulling on the blood vessel wall by the stent, rather than merely pushing at major-axis ends of the stent frame, as described above. For example, in some implementations, circle-to-oval reshaping of a target blood vessel is implemented by a non-circular stent by pushing radially outwardly against the major-axis ends of the blood vessel to thereby cause radially-inward deflection of the minor-axis walls of the blood vessel to produce the oval shape thereof. Due to physical coupling between the minor-axis walls of the non-circular stent and the associated blood vessel wall segments, facilitated by tissue-coupling elements/features as disclosed herein, as the stent elongates in the major-axis dimension, as described in detail herein, the minor-axis walls of the stent can radially-inwardly pull the blood vessel walls to expedite/improve the reshaping of the blood vessel to more closely matched that of the reshaping of the stent segment.

When a blood vessel is reshaped exclusively through major-axis pushing on the blood vessel, a relatively large force may be required to reshape the blood vessel to the desired oval shape. However, where tissue-engagement features are implemented in a manner as to provide for pulling on the tissue wall as well pushing, relatively less major-axis pushing/stretching may be required to achieve comparable blood vessel volume change/reshaping due to the ability to collapse the minor-axis blood vessel walls radially inwardly from the inside using the stent frame. Coupling the stent frame to the blood vessel can reduce gaps between minor-axis blood vessel walls and stent frames, which can be advantageous as adhesion between the stent frame and the blood vessel may be impeded and/or absent in the presence of free gaps between such structures.

FIG. 11 shows a non-circular stent 600 with tissue-engagement features 71 in accordance with one or more examples. In order to couple a target blood vessel to the stent frame 631 of the stent implant 600, the implant 600 includes certain tissue-engagement features 71, as described in detail herein, such as hooks, barbs, spikes, and/or the like. The implant 600 may or may not have a covering 645 thereon. For example, the implant may include a stent frame 631 having in outer (or inner) cover 645, wherein the tissue-engagement features 71 of the stent frame may be designed/configured to puncture and/or protrude through the covering 645 at least in part to provide for direct physical contact between the tissue-engagement features 71 and the outer vascular tissue. The tissue-engagement features 71 may comprise spikes, barbs, or the like, that project radially outwardly, axially/longitudinally, and/or at any angle therebetween with respect to the axis Aₛ of the stent implant.

In some implementations, a series of anchor elements 71, such as spikes, barbs, or the like, can extend radially away from the stent frame 631, wherein such tissue-engagement features 71 are configured to engage with the surrounding blood vessel wall when the stent frame is expanded and compressed thereagainst. Once the tissue-engagement features 71 have been pressed against and/or into the blood vessel tissue, coupling/engagement between the features 71 and the blood vessel wall may be retained as the stent subsequently transitions between an expanded, more-circular state and a free/biased non-circular/oval state.

The tissue-engagement features 71 may be associated with any axial portion/segment of the implant device 600. For example, the medial segment 620 between sealing end segments 640 may have the tissue engagement features 71 associated therewith, whereas the circular end segments 640 may not have tissue engagement features. For example, due to the expanded circular shape of the end segments 640, secure fixation of the end segments 640 to the blood vessel may be achieved through radial expansion force and/or friction forces around substantially the entire circumference of the frame. The medial segment 620, on the other hand, may not have a biased circular shape, and therefore minor-axis walls 625 thereof may generally not expand outwardly into full physical contact with the blood vessel wall. Therefore, tissue-engagement features 71 can advantageously hold the blood vessel wall to the stent frame 631 in such segments. Although the stent implant of FIG. 11 is shown as a circle-oval-circle stent, it should be understood that tissue engagement features may be implemented with non-circular stent/stent segments having any suitable or desirable configuration, including stents that have a common shape throughout the length thereof and do not include differently-shaped sealing end segments.

The tissue-engagement features 71 may extend mainly at the level of the engagement frame 631 of the implant, such that they are configured to frictionally interact with the surrounding blood vessel tissue. Additionally or alternatively, tissue-engagement features of the implant may comprise spikes, barbs, or similar features that are angled and/or extend radially outwardly so as to facilitate penetration into surrounding blood vessel tissue. As referenced above, the tissue engagement spikes, barbs, or other similar features 71, may be implemented over any axial and/or circumferential portion of the stent frame with respect to the axis Aₛ thereof. Furthermore, the tissue-engagement features 71 can have any suitable or desirable physical structure/configuration.

Referring to example detail 601, which shows example implementations of the tissue-engagement features 71, the tissue-engaging elements 71a are projections (which can also be referred to as ‘spikes,’ ‘barbs,’ ‘anchors,’ or similar) extending from the struts 638 of the stent frame 631. The projections 71a are configured to engage (and in some instances penetrate) the native blood vessel tissue. In this manner, the projections 71a can increase the frictional engagement between the stent 600 and the blood vessel, particular in the area of the non-circular segment 620 of the stent 600. The projections 71a can also help to improve tissue ingrowth.

The projections 71a can extend in various directions from the struts 638 of the stent frame 631. For example, some of the projections 71a can extend from the struts 638 at an angle relative to a central longitudinal axis Aₛ extending from the inflow end to the outflow end of the stent 600. In some instances, the projections 71a are perpendicular or at least substantially perpendicular (e.g., forming an angle of 80-100 degrees) to the struts 638 from which they extend. In other examples, the projections 71a can extend from their respective struts at various other angles (e.g., between 1-79 degrees). The projections 71a can comprise various shapes and lengths such that the projections 71a provide sufficient retention force for the stent walls 625, while reducing potential harm to the surrounding tissue. For example, the projections 71a can comprise tines or spikes. In other examples, the projections 71a can comprise ball-shaped bulges and/or a rectangular shape. Additionally or alternatively, one or more of the projections 71a can comprise a curved shape, a hook shape, a cross shape, a T-shape, and/or a barbed shape. Various combinations of shapes and/or sizes of projections can be used. The projections 71a may be similar to any of the example tissue-engagement features shown in example detail images 607, 608, and 609, which show tissue-engagement features projecting in an axial dimension with respect to a stent frame axis.

With reference to detail image 602, example implementations of the tissue-engagement features 71 of FIG. 11 can include radially-extending/projecting projections/spikes 71b. The tissue-engagement projections 71b, in addition to projecting radially outwardly, may deflect distally or proximally. The projections 71b may be similar to any of the example tissue-engagement features shown in example detail images 603, 604, 605, and 606, which show tissue-engagement features projecting in radial and/or distal/proximal dimensions with respect to a stent frame axis.

Example detail images 603-609 show different arrangements of spike-type tissue-engagement features 71 extending from stent frame struts 638, illustrated in side/sectional views. Images 603-605 show variations of angled spikes 71, extending from struts 638 of the stent frame 631, including struts from which project distally-deflecting spikes 71c, proximally-deflecting spikes 71d, and both distally-71e and proximally-71f deflecting spikes.

The angled tissue-engagement spikes 71c, 71d, 71e, 71f can extend at an angle θ from parallel with the stent axis Aₛ, such that the spikes project radially outward and in an axial direction. In some examples, the angle θ is in the range of 10-80 degrees. In some examples, the angle θ is in the range of 20-70 degrees. In some examples, the angle θ is in the range of 30-60 degrees. Implementations in which struts 638 include both distally-extending spikes 71e and proximally-extending spikes 71f as in detail 605 can be advantageous in that the oppositely-oriented spikes of such struts can improve engagement with the tissue wall. The angled spikes may be considered to be angled or deflected with respect to a plane of the frame 631 and/or with respect to an axial and/or circumferential dimension/plane of the frame 631. Furthermore, whereas some implementations of tissue-engagement projections, such as the tissue engagement projection 71g in image 606, project radially outwardly in a dimension that is normal/orthogonal to a surface or plane of the stent frame 631, angled tissue-engagement projections, such as in images 603-605, may be considered angled with respect to a dimension normal to the stent frame surface/plane.

Example detail images 607-609 show different arrangements of spikes that extend solely in the axial direction (i.e., parallel to central axis Aₛ) and not in a radial direction. For example, both distally-extending spikes 71h and proximally-extending spikes 71i extend solely in the axial direction, such as between the inner and outer surfaces of the stent frame 631.

The tissue-engagement anchors 71 may comprise hooks that have curvature designed to cause the anchors to penetrate the blood vessel wall and pull back on the vessel wall towards the stent frame. That is, the anchors 71 may comprise features that facilitate embedding and grabbing/pulling on the vessel wall. The tissue-engagement anchors 71 may be configured to penetrate into the tissue without fully penetrating through the blood vessel wall, thereby preventing a through-and-through puncture/perforation in the blood vessel wall. The anchors 71 may comprise curved spikes configured to penetrate in and out of the blood vessel wall. In some implementations, the anchors have a harpoon-type shape, such as an arrow structure configured to be embedded within the blood vessel tissue while impeding withdrawal/retraction from the blood vessel wall once punctured. The tissue-engagement anchors/features 71 may advantageously be relatively minimalistic so as to not undesirably compromise the integrity of the wall of the target blood vessel. Therefore, the number of anchors implemented may be selected to reduce the number of perforations/punctures in the blood vessel wall necessary to hold the blood vessel to the stent frame.

The tissue-engagement anchors/features 71 may be implemented on any area of the stent frame 631, including any segment(s) along the axial length of the frame 631 and/or any area around a circumference/perimeter of the stent frame 631. For example, the anchors 71 may be implemented primarily or exclusively on the non-circular portion 620 and/or the non-circular 620 and transition 650 portions, or portions thereof. Furthermore, the anchors may be implemented primarily or exclusively on the minor-axis wall portions 625 rather than the major-axis end walls 627.

In order to cause the tissue-engagement features 71 of the implant 600 to penetrate and/or otherwise engage/embed within the target blood vessel wall, may be necessary or desirable to reshape the non-circular medial portion/segment 620 to a circular shape to facilitate physical contact between the stent frame 631 in such segment 620 with the blood vessel wall. For example, circularizing the medial/non-circular stent segment 620, in implementations in which the perimeter of the stent segment 620 is matched to and/or exceeds the perimeter of the blood vessel wall, can cause the tissue-engagement features 71 to be physically pushed against the inner diameter of the blood vessel, thereby facilitating engagement of the anchoring elements 71 with the blood vessel.

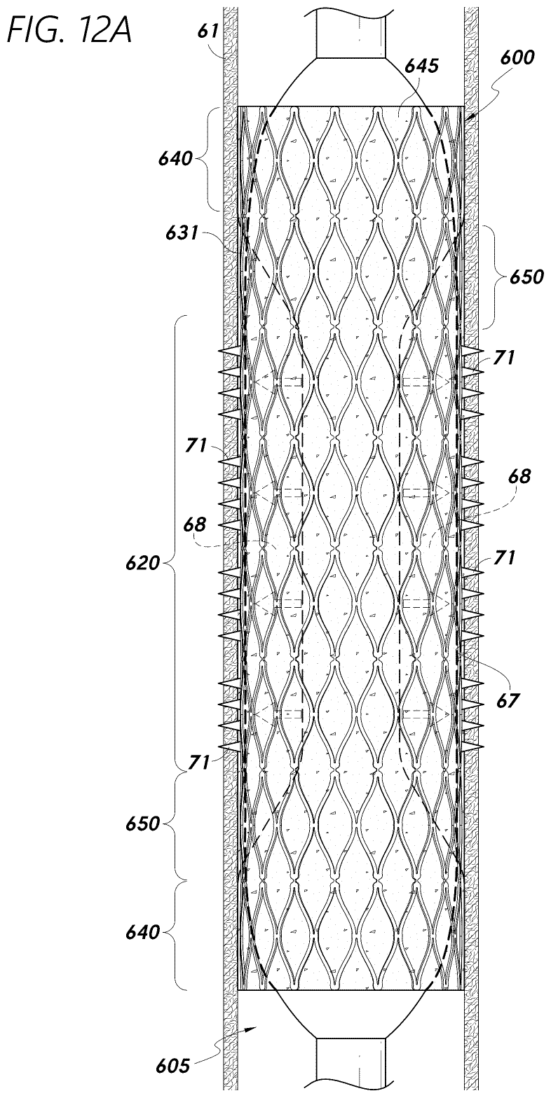

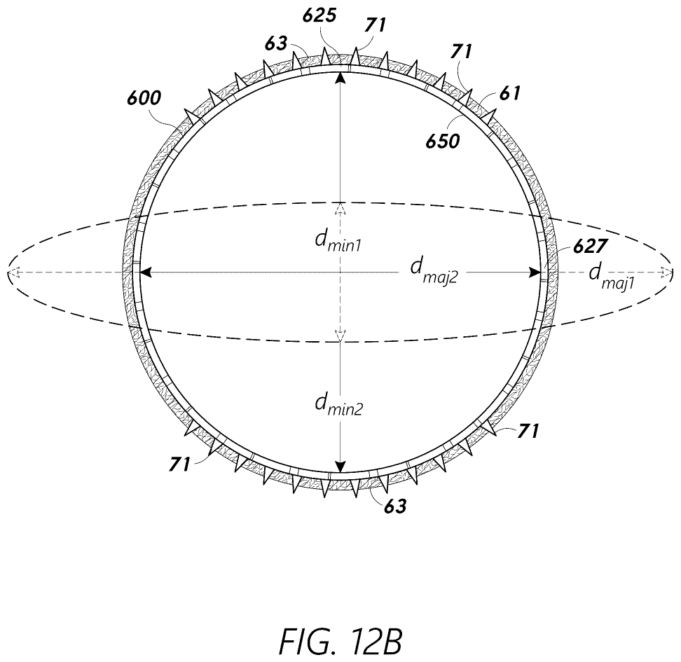

FIGS. 12A and 12B show the stent implant 600, having tissue engagement spikes/features 71 associated at least with the non-circular segment 620 thereof, disposed within a blood vessel 61 with the non-circular portion 620 thereof circularized to engage the tissue-engagement features 71 with the blood vessel wall 61 in accordance with one or more examples.

The non-circular stent 600 can be expanded into contact with the blood vessels inner wall, thereby facilitating engagement of the anchoring elements 71 therewith. For example, the expansion of the non-circular stent segment 620 can cause the tissue-engagement features/anchors 71 to penetrate into the vascular wall. Expansion of the non-circular/oval stent segment 620 from its free/biased non-circular state to the expanded, more-circularized state shown in FIGS. 12A and 12B can be accomplished through any means or mechanism. For example, a balloon inflation or other expansion device 605 (e.g., balloon catheter) may be inflated/expanded within the inner diameter/channel of the stent segment 620 to force the stent 600 to reshape to a more-circular shape, thereby expanding the minor-axis diameter thereof (e.g., from the smaller diameter dₘᵢₙ₂ to the expanded diameter dₘᵢₙ₂). In some implementations, a temporary stent may be expanded with in the stent channel to force the circular state thereof, wherein the expansion stent may be collapsed for retrieval after the tissue of engagement features/anchors 71 have become coupled to the blood vessel wall 61. In some implementations, a stent expansion/circularizing device may be configured to degrade over time, thereby allowing the stent to revert to its biased non-circular shape after degradation/removal of the circularizing structure (e.g., stent, tie, or other structure designed to force a more-circular shape of the stent segment 620). As tissue-engagement features/anchors (e.g., spikes) may be configured to retain engagement with the blood vessel wall, such that when the stent reverts back to its oval-shaped configuration as shown in FIG. 11, the vascular wall 63 may be pulled radially inwardly therewith, with relatively minimal or no gap between the stent frame 631 and the blood vessel wall in which blood can be trapped/collected.

Circularization of the non-circular stent segment 620 to expand such segment against the inner diameter of the blood vessel wall 63 may advantageously force any trapped/collected blood present in the gap area 68 out from around the stent frame 631, wherein space previously occupied by the collected blood may be occupied by the blood vessel wall 63, thereby eliminating, at least in part, the gap areas 68 between the stent frame 631 and the blood vessel wall 63.

Although illustrations of the present disclosure show tissue-engagement features that are integrated with the stent frame of a stent implant device, in some implementations, the tissue engagement features/anchors can be part of a separate frame that can be attached (e.g., sutured) to the stent. Furthermore, the tissue engagement elements/anchors can be disposed in any suitable arrangement around the stent, including arrangement in a spiraling manner, or other configuration that may facilitate proper/desirable coupling between the stent frame and the blood vessel wall. While tissue-engagement features of examples of the present disclosure are illustrated as spikes or similar structures in some contexts of the present disclosure, other anchoring structures may be utilized that are configured to retain engagement with the surrounding vascular tissue during transitioning of the stent between an expanded state and an oval/non-circular free state.

The expansion of the stent segment 620 as shown in FIGS. 12A and 12B provides the ability to anchor the tissue-engagement features/anchors 71 to the blood vessel wall, which may be necessary due to the stent segment 620 having a non-circular shape. In order to make sure there is full contact between the blood vessel 61 and the stent anchors 71, a balloon catheter 605 having a circular cross-section (balloon catheter 605 not shown in FIG. 12B for visual clarity) may be expanded within the stent 600 to force the stent 600 to a circular shape such that the outer diameter of the stent segment 620 touches the inner diameter 67 of the blood vessel 61, creating sufficient contact tanker to the vessel wall. Although described as an oval stent segment, it should be understood that any other non-circular shape of the stent segment may be implemented, such as peanut-shaped as described in detail herein. In some implementations, circularization/expansion of the non-circular segment 620 is achieved by pulling major-axis ends/sides 627 of the stent frame 631 in the non-circular segment 620 radially inward to thereby cause outward deflection of the minor-axis sides/walls 625. A tie may be implemented across the major axis of the segment 620 to hold the stent in the more-circularized shape. For example, the tie may be secured to the stent frame 631 prior to deployment from a delivery system/catheter, such that deployment of the stent 600 involves initially expanding the frame 620 in the more-circular shape until the tie is broken, dissolved, or otherwise removed.

The stent frame 631 may be covered on an inner diameter and/or an outer diameter thereof with a fluid-impeding (e.g., fluid-tight) covering 645. However, it should be understood that in some implementations, example stent implant devices comprising tissue-engagement features may be implemented without coverings. In such examples, the coupled blood vessel wall, which may be held against the stent frame via tissue-engagement features as described herein, may serve as a covering for the stent implant that channels blood flow through the channel of the stent implant. Any cover or covering disclosed herein may be at least partially fluid-impeding in that fluid (e.g., blood) cannot flow freely therethrough, and in some instances, covers/coverings disclosed herein are entirely fluid-blocking/tight.

Depending on the relative size of the stent segment 620 to the blood vessel segment that is spanned thereby, certain circumferential wrinkles/bulges may form in the blood vessel when anchored to the stent frame 631. In some implementations, oversizing of the stent segment 620 with respect to a circumference/perimeter dimension thereof may be implemented to reduce the amount of gaps/wrinkles that form when coupling the stent frame to the blood vessel wall.

The pulling effect of the stent frame 631 on the blood vessel wall after coupling between the tissue-engagement anchors and the blood vessel wall has been achieved can require relatively secure/deep penetration of the anchors 71 into the tissue wall due to the thickness thereof. Once the expansion force is removed that was implemented to force the stent segment into contact with the blood vessel wall, the stent 600 may be permitted to collapse/revert to its non-circular free state and pull the blood vessel wall 63 with the minor-axis walls 625 of the stent segment.

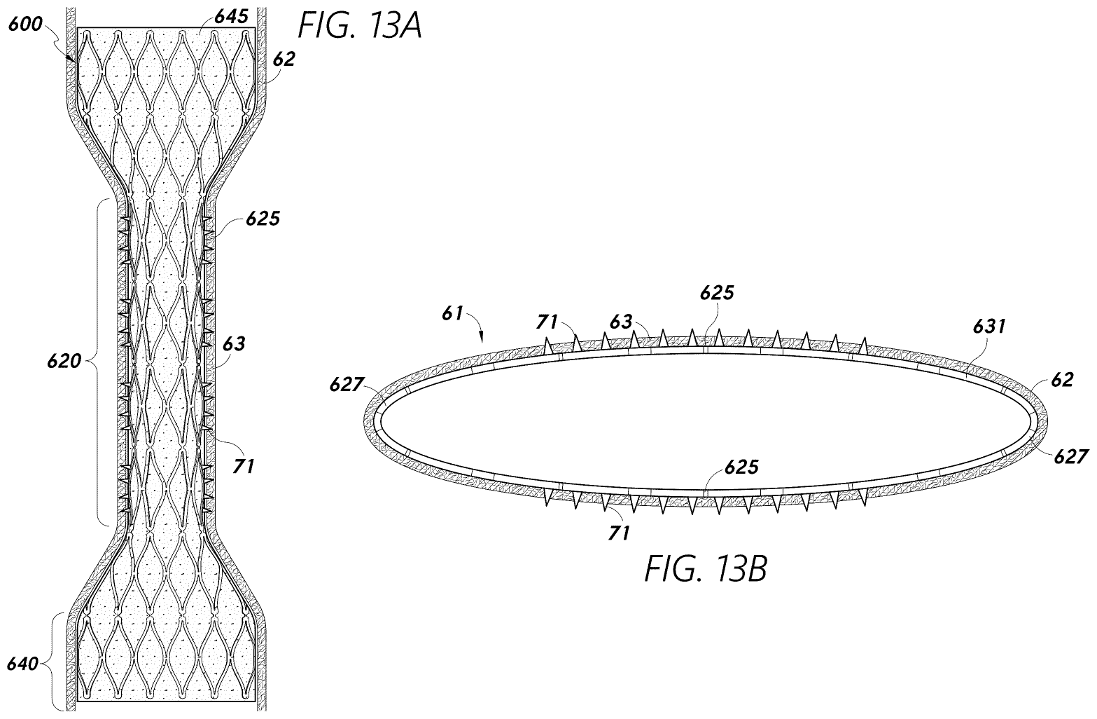

FIGS. 13A and 13B show the non-circular stent 600 disposed within the blood vessel 61 with the tissue-engagement features 71 of the non-circular portion 620 of the stent frame 631 engaged with the blood vessel wall in accordance with one or more examples. That is, the state shown in FIGS. 13A and 13B may correspond to the stent implant 600 after engagement/coupling of the tissue-engagement features/anchors 71 with the blood vessel wall and removal of any expansion force (e.g., balloon catheter, etc.), such that the stent segment 620 is returned to a non-circular biased shape/state thereof (e.g., oval- or peanut-shaped), wherein the blood vessel walls 63 have been pulled radially inwardly with the stent frame walls 625. The axial view of FIG. 13B shows the blood vessel 61 in the area of the non-circular segment 620 of the stent frame 631 wherein the blood vessel 63 is in relatively close contact with the outer diameter of the stent frame 625 and being held/coupled thereto by the tissue-engagement anchors 71. With the blood vessel walls 63 held in contact with the stent frame 625, tissue overgrowth may be facilitated between the blood vessel wall and the frame 631 and/or covering feature 645 associated therewith.

### Additional Stent Anchoring Concepts

In some examples of the present disclosure, tissue-engagement anchors are implemented in connection with stent frames, wherein the tissue anchors are integrated with the stent frame itself. However, it should be understood that such examples, some of which are described above, may include anchors that are separable and/or distinct in some manner from the structure of the stent frame. For example, the anchors may be associated with the cover of the implant device or other structure that is deployed in an assembly with the stent and/or stent frame. Furthermore, as described in detail above, in some implementations, tissue-engagement anchors may be embedded in blood vessel tissue together with the expansion or deployment of the stent frame itself. Alternatively, tissue-engagement anchors configured to secure a stent frame to blood a vessel wall may be deployed and/or engaged with the blood vessel as separate components and/or in accordance with a separate process or sub-process. For example, in some examples, the present disclosure provides for deployment of helical tissue anchors (e.g., corkscrew anchors), or other types of tissue anchors, to secure the stent frame to a blood vessel after deployment/placement of the stent/stent-frame in the target blood vessel. In some implementations, a series of tissue anchors may be deployed around an oval or other non-circular stent segment, which may be cinched via a wire, suture, or other tie or mechanism when engaged with the blood vessel wall, thereby forming a relatively tight attachment of the stent to the surrounding vascular wall.

FIGS. 14-1, 14-2, 14-3, 14-4, and 14-5 illustrate a flow diagram for a process 1400 for engaging a non-circular portion of a stent with a blood vessel wall using anchors in accordance with one or more examples. FIGS. 15-1-15-14 provide images of aspects of the stent 700, tissue anchors 75, delivery system components, and anatomy relating to operations of the process of FIGS. 14-1, 14-2, 14-3, 14-4, and 14-5 according to one or more examples.

At block 1402, the process 1400 involves deploying a stent 700 having a non-circular segment in a target blood vessel segment 61. The stent 700 may be a non-circular stent in accordance with any example disclosed herein. For example, the stent 700 implant may have a non-circular medial segment 720 and circular end segment(s) 740, as described herein in connection with some examples, or may be non-circular with respect to a biased/free shape thereof along substantially the entire length of the stent. The process 1400 may be implemented to deploy a series of helical anchors, for example, or other types of anchors, which may be screwed into the vascular wall surrounding the deployed stent. The stent 700 may be advanced to the target deployment site in a crimped state/configuration within the delivery system sheath/catheter.

FIG. 15-1 one shows an example stent 700a deployed within a target blood vessel 61, wherein the stent has a non-circular shape. The view of FIG. 15-1 shows the minor-axis dimension of the stent, which may be lesser than the major-axis (e.g., out of the page with respect to the orientation of FIG. 15-1) dimension of the stent. As described in detail above, certain gaps 68 may initially be present between the minor-axis stent sides/walls 725 and the inner diameter of the blood vessel 61 in such areas. For example, such gaps 68 may form due to the major-axis stretching/pushing-out of/on the blood vessel 61, such as at opposite walls/sides 62 of the blood vessel 61, which may stretch the blood vessel 61 and cause some amount of inward deflection of minor-axis walls 63 thereof; such deflection may not fully hug/hold-to the stent frame minor-axis walls 725, but rather may remain separated therefrom by a gap distance, producing the gaps 68.

FIG. 15-2 shows an alternative stent implant device 700b implanted in the target blood vessel 61, wherein the stent 700b has a non-circular medial segment 720 and circular end segment(s) 740, which design may be similar in one or more respects to any of the stent implant devices disclosed herein comprising sealing end segment(s) and a non-circular medial segment. In the example of FIG. 15-2, the gaps 68 may initially be formed in the area outside of the medial segment 720 of the stent 700b. In some cases, at least initially, the gaps 68 may be filled with blood, which may become trapped in the areas outside the medial segment 720 of the implant due to the ends 740 of the stent implant 700b being substantially sealed against the inner blood vessel wall and preventing outflow from the gap spaces 68. FIG. 15-3 shows an axial view showing an example representation of the non-circular stent segments of either the of the embodiments 700a, 700b deployed in the blood vessel 61 with the gap areas 68 present between the minor-axis walls 725 of the stent 700 and the walls 63 of the blood vessel 61.

At block 1404, the process 1400 involves introducing an anchor delivery tool 72, as a catheter or similar device, within the lumen/channel 749 of the stent 700. The anchor delivery tool 72 may be used to deploy one or more tissue anchors 75 into the blood vessel wall 61 for the purpose of securing the minor-axis walls 725 of the stent 700 to the blood vessel wall. Initially deploying the stent 700 from a delivery system may allow the stent 700 to assume oval or other non-circular biased/free state. The anchor tool 72 may be advanced along the stent's centerline to a target position therein. In some implementations, the stent comprises a cover 745, wherein penetration of the anchor 75 through the cover 745 prior to engagement with the blood vessel wall can reduce the risk of aortic/vessel perforation due to the presence and positioning of the covering 745 of the implant.

FIG. 15-4 shows a side view of the stent 700 deployed in the blood vessel, wherein the anchor-delivery tool 72 is advanced to a position within the non-circular segment of the stent 700. The illustrated segment of the stent 700 shown in FIG. 15-4, as with any other illustrated stent segment of the present disclosure, may represent a naturally non-circular segment of the stent 700a or FIG. 15-1 or the stent 700b of FIG. 15-2.

At block 1406, the process 1400 involves deploying the one or more anchors 75 through a first side 725 of the stent frame 731 and at least partially into a corresponding portion 63 of the blood vessel wall. To deploy the tissue anchor 75, the anchor tool 72 may be bent, for example by approximately 90°, to allow for the distal end of the tool 72 to project radially outward toward the stent frame 731, such that the distal end of the anchor tool 72 is pressed against the inner diameter of the stent 700. A helical anchor or other type tissue anchor 75 may be screwed or otherwise extended out of the shaft of the tool 72 and through the stent frame 731 and/or cover 745 toward the blood vessel wall 63. Further rotating or otherwise advancing the tissue anchor 75 (e.g., helical anchor) using a torque tube or other torque-transmitting driver/device 74 may serve to screw the tissue anchor 75 into the blood vessel wall 63. In some implementations, the driver 74 may then be withdrawn/retrieved, leaving a suture 79 coupled to the anchor, wherein the suture/tie/wire may be used to singed the blood vessel wall to the stent frame.

FIG. 15-5 shows the delivery tool 72 deflected/articulated to face in a radial orientation with respect to an axis Aᵥ of the stent 700 to allow for deployment/advancement of the tissue anchor 75 therefrom radially outwardly towards the blood vessel wall 63 and into engagement/embedding with the blood vessel wall 63. The tissue anchor 75 may comprise, for example, a helical/corkscrew tissue-engagement form and/or a driver head component. For example, the anchor 75 may be embedded into the blood vessel wall 63 by rotating the anchor 75 in accordance with a chirality thereof to thereby drive the tissue-engagement portion of the anchor 75 into the blood vessel tissue. FIG. 15-6 shows an axial view of the tool 72 being used to advance and/or embed the tissue anchor 75.

As illustrated in the example of FIGS. 15-5 and 15-6, a driver tool 74 may be used to advance the tissue anchor 75 from the catheter 72 to the blood vessel wall 63. For example, the inner driver 74 may be advanced, along with the tissue anchor 75, through at least a portion of the frame 731 and towards the blood vessel wall by traversing the space 68 between the frame 731 and the blood vessel 61. In some implementations, screwing/rotating of the anchor 75 may be necessary to pass the tissue engagement portion thereof through the stent frame 731 by threading the anchor 75 through a gap/cell 735 in the stent frame 731.

FIG. 15-7 shows an alternative implementation in which the anchor 75c does not passing entirely through the stent frame 731, but rather a proximal head/driver portion 76 thereof may remain and/or be disposed within the inner diameter 749 of the stent frame 731 when advancing the tip of the anchor 75c towards and into the blood vessel wall 63. The anchor 75c may be embedded in the tissue 63, for example, by rotating the drive head 76 of the anchor 75c such that a tissue-engaging element/portion 73 of the anchor 75c becomes driven into the tissue 63. The tissue-engaging element/portion 73 of the anchor 75c may have a corkscrew form, as illustrated.

The driver tool 74 may be used to rotate the driver head 76 within the channel 749 of the stent frame 731, which may cause the tip of the tissue-engagement portion 73 of the anchor 75c to embed in the blood vessel wall 63 and, as the helical screw/anchor 75c is rotated, may draw the blood vessel wall 63 towards the outer diameter of the stent frame 731, thereby coupling the blood vessel wall 63 in closer position/contact with/to the stent frame 731. The screw anchor 75c can be rotated in-place to draw the tissue 63 towards the stent frame 731 once the tissue 63 has been caught by the tissue-engagement tip/portion 73 of the anchor 75c. For example, the tissue anchor 75c may be disposed at a fixed position relative to the stent frame.

With respect to the various examples shown, in some implementations, driving the tissue anchor 75 into the blood vessel wall 63 may cause the tip of the anchor to puncture through the blood vessel to an exterior of the blood vessel 61 and project by some distance from the blood vessel. For example, the tip of the tissue anchor 75 may extend two, three, four, or more millimeters out from the vessel wall. In some implementations, the protrusion of the tip of the tissue anchor 75 from the blood vessel wall may be maintained at a distance less than approximately 2 mm to avoid interference with anatomy outside of the target blood vessel, such as within the abdominal cavity. Minimizing the projection distance of the tip of the anchor 75 in implementations in which the anchor passes fully through the thickness of the blood vessel wall 63 can prevent penetration or damage to other organs or and/or tissues.

At block 1408, the process 1400 involves cinching the blood vessel wall 63 to the side 725 of the stent, to thereby approximate the blood vessel wall 63 closely to the outer diameter of the stent frame 731. For example, in some implementations, the delivery system 72 may be implemented with a suture or other connector/tie 79 coupled to the anchor 75, such as to a proximal portion thereof, wherein the suture/tie passes from the tissue anchor 75 into the delivery tool 72 through the stent frame 731 after engagement of the anchor 75 with the blood vessel wall 63. In such implementations, drawing the blood vessel wall 63 to the stent frame 731 may involve pulling/tensioning and/or otherwise drawing the suture/tie to the stent frame 731. In some implementations, the suture/tie 79 may be fixed/coupled to a proximal portion/end of the anchor 75, and the anchor 75 may be pulled to the stent frame 731 by pulling the proximal portion of the anchor 75 via the suture/tie 79.

Once the anchor 75 has been pulled/cinched to the stent frame 731, the process 1400 may involve fixing or securing the tension of the suture 79 and/or otherwise tying or securing the anchor 75 to the stent frame 731 in a permanent fixation. Cinching of the deployed tissue anchor 75 to the stent 731 may serve to relatively-tightly attach the blood vessel wall 63 to the stent 700 to allow the blood vessel wall 63 to move with the wall 725 of the stent 700 when the stent 700 transitions between expanded/circular and compressed/non-circular states. Cinching the blood vessel wall 63 to the stent frame 731 may expel blood from around the stent frame 731 that may otherwise collect in the gap area 68 between the stent frame 731 and the blood vessel 61, thereby reducing thrombus risk and increasing the efficacy of blood vessel reshaping as described in detail herein. In some implementations, the stent segment 700 may be forced to a circular shape to force contact between the stent frame 731 and the blood vessel wall 63 prior to deployment of tissue anchor(s) 75, which may advantageously facilitate engagement between the tissue anchor 75 and the blood vessel segment 63 without having to first span a gap 68 between the stent frame 731 and the blood vessel.

FIG. 15-8 shows the suture/tie 79 coupled to the anchor 75 and passing into the delivery tool 72, whereas FIGS. 15-9 shows the anchor 75 having been pulled/cinched to the stent frame 731 using the suture/tie 79. FIG. 15-10 shows an alternative implementation in which cinching/pulling the blood vessel wall 63 to the stent frame 731 involves rotating the helical tissue anchor 75c from within the channel 749 of the stent frame 731, as in accordance with the example of FIG. 15-7. In such implementations, no suture/tie may be utilized or necessary to cinch/draw the blood vessel wall 63 to the stent frame 725. In some implementations, the tie/line 79 can be drawn proximally through eyelet or other line-engagement/coupling feature(s) associated with a proximal end/head 76 of the anchor 75/75a to secure the anchor to the tie/line 79.

With respect to any of the illustrations shown, cinching the blood vessel wall 63 to the stent frame 731 may serve to expel blood that has collected in the space 68 between the stent frame 731 and the blood vessel wall 63 on the side of the frame 731 where the tissue anchor 75 has been deployed. In some implementations a bead, clip, or other locking means/mechanism may be placed on the suture/tie 79 to hold the tension therein and maintain the tissue anchor in secure coupling with the stent frame 731 after deployment. The suture/tie 79 may be cut once the desired tension/distance has been achieved.

At block 1410, the process 1400 involves deploying a second/subsequent tissue anchor 75b through a second side 725b of the stent 700 and into a blood vessel wall 63b opposite of the first deployed tissue anchor 75a. For example, the delivery tool 72 may be used to advance the tissue anchor 75b through the opposite minor-axis side 725b of the stent frame 731 for tissue anchor deployment and blood vessel wall cinching for the opposite minor-axis wall 725b of the stent 700. The second tissue anchor 75b may be deployed using the same delivery tool 72 as with the first tissue anchor 75a, or a separate tool may be introduced for the purpose of deploying the second anchor 75b and/or one or more anchors on the second side 725b of the stent 700. FIG. 15-11 shows the first tissue anchor 75a secured/coupled to the stent frame 631, with the second anchor 75b being embedded in the blood vessel wall 63b.

While some of the images described above show tissue anchors deployed on opposite minor-axis sides of a non-circular stent implant, it should be understood that tissue anchors may be deployed at any angular position with respect to the axis of the stent. In some implementations, it may be desirable to initially deploy tissue anchors in circumferential/perimeter portions of the stent frame that are relatively close to the major-axis ends of the stent, due to the proximity of the blood vessel wall to the stent frame in such areas. Therefore, tissue anchors may initially be deployed in such areas, where the gap distance between the stent frame and the blood vessel wall may be relatively minimal, such that the tissue anchor may more readily/easily reach and penetrate the blood vessel wall from/through the stent frame, as compared to deployment in the area of the minor-axis of the stent, which may be associated with a greater gap distance between the stent frame and the blood vessel wall in some cases.

FIG. 15-12 shows an example implementation in which a first tissue anchor 751 is deployed through the stent frame 731 and into the blood vessel wall 61 in an area proximate to the major-axis end 727 of the stent frame 731. Deployment of the first tissue anchor 751 may draw the blood vessel wall 61 closer to the stent frame 731, thereby allowing for a subsequent tissue anchor 75₂ to be deployed in an area farther removed circumferentially from the major-axis end 727 of the stent frame 731, wherein the gap between the stent frame 731 and the blood vessel wall in such area may be reduced through the coupling of the first tissue anchor 75₁ with the blood vessel wall 61, thereby facilitating easier deployment of the second tissue anchor 75, into the blood vessel wall 61 as the blood vessel wall has been drawn relatively closer to the stent frame 731.

FIG. 15-12 further shows a third tissue anchor 751 at the minor-axis area of the stent frame 731, which may be deployed subsequent to the second tissue anchor 75₂, after the blood vessel wall 61 has been brought relatively closely to the stent frame 731 through cinching of the second tissue anchor 75₂, thereby facilitating deployment of the third tissue anchor 753 into the blood vessel wall 61. Although tissue anchors are shown only between one major-axis end 727 of the stent 700 and the minor axis of the stent, it should be understood that tissue anchors may be deployed sequentially moving from the major-axis sides to the minor-axis portion of the stent frame from both sides in some implementations. Circumferentially-sequential tissue anchor deployment as shown in FIG. 15-2 may be particularly useful for implementations utilizing tissue anchors that are driven/rotated from within the stent frame, such as in the examples of FIGS. 15-7 and 15-10.

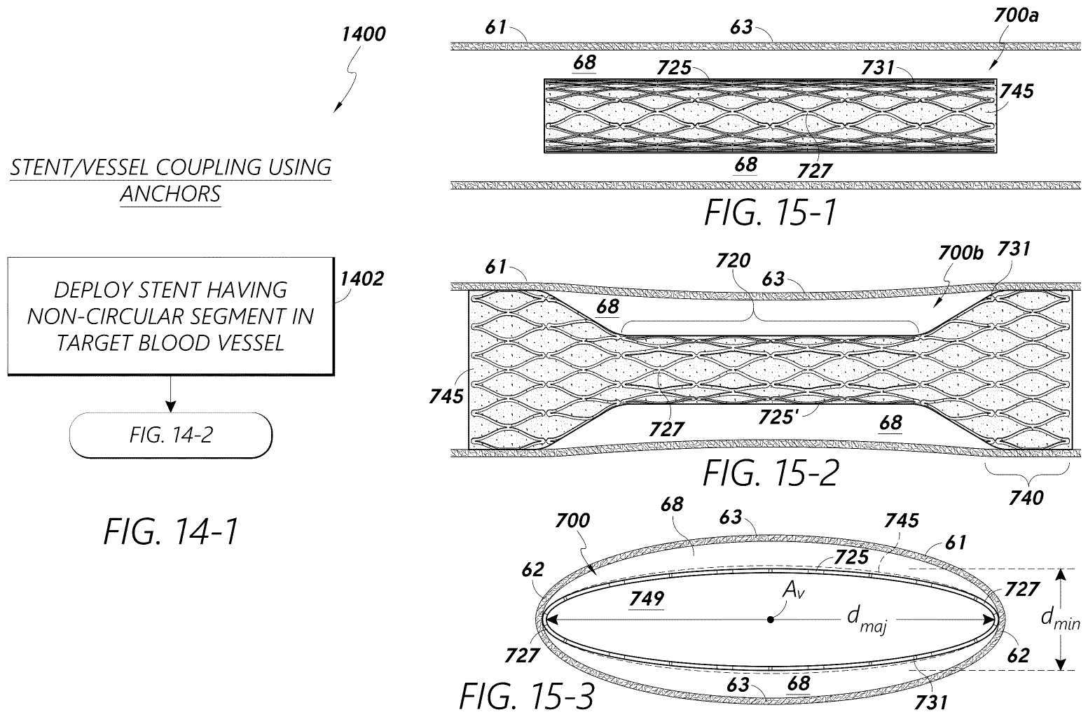

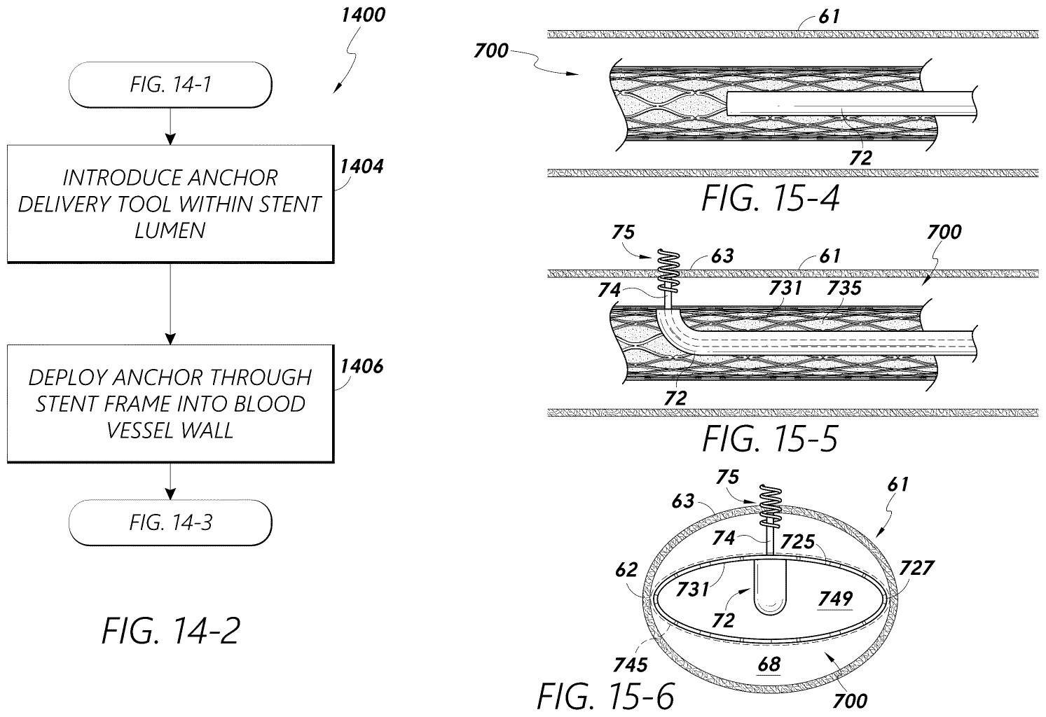

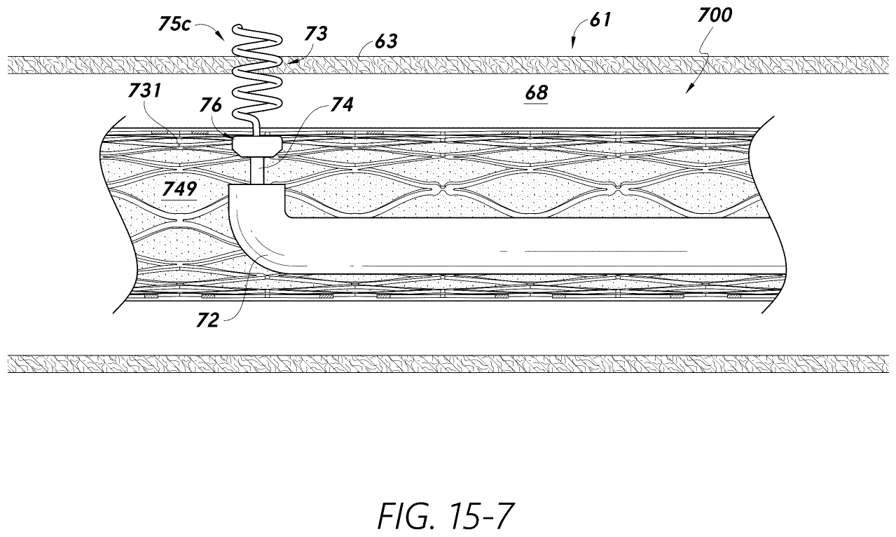

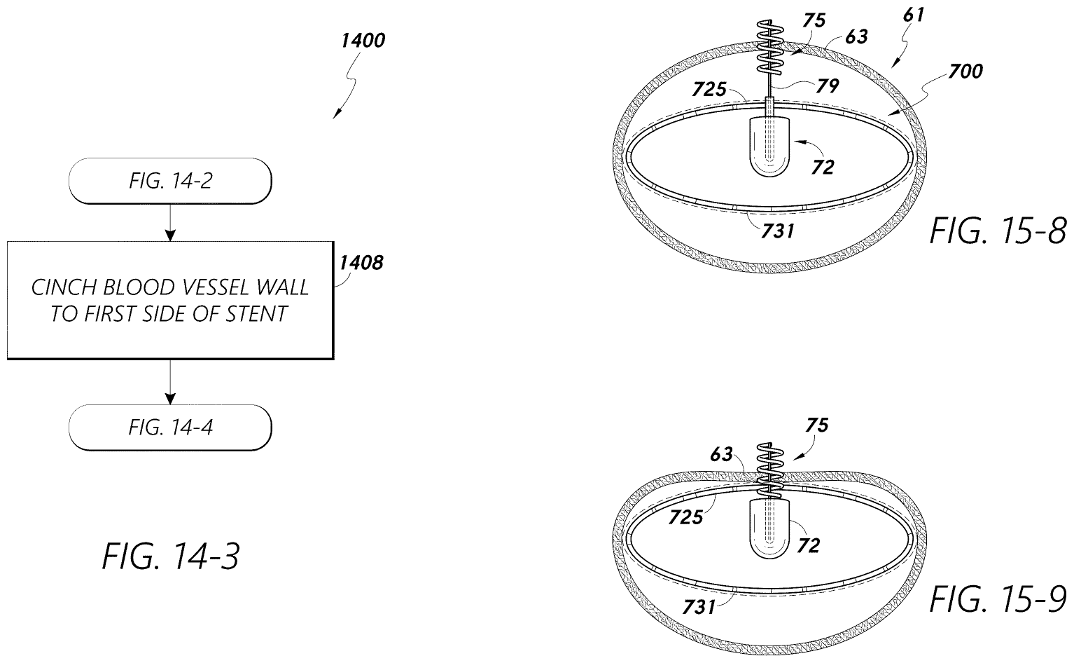

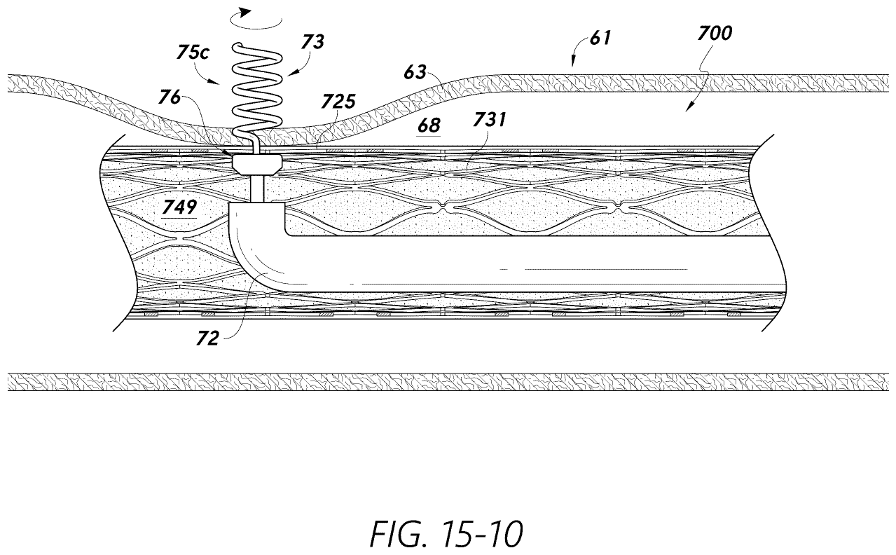

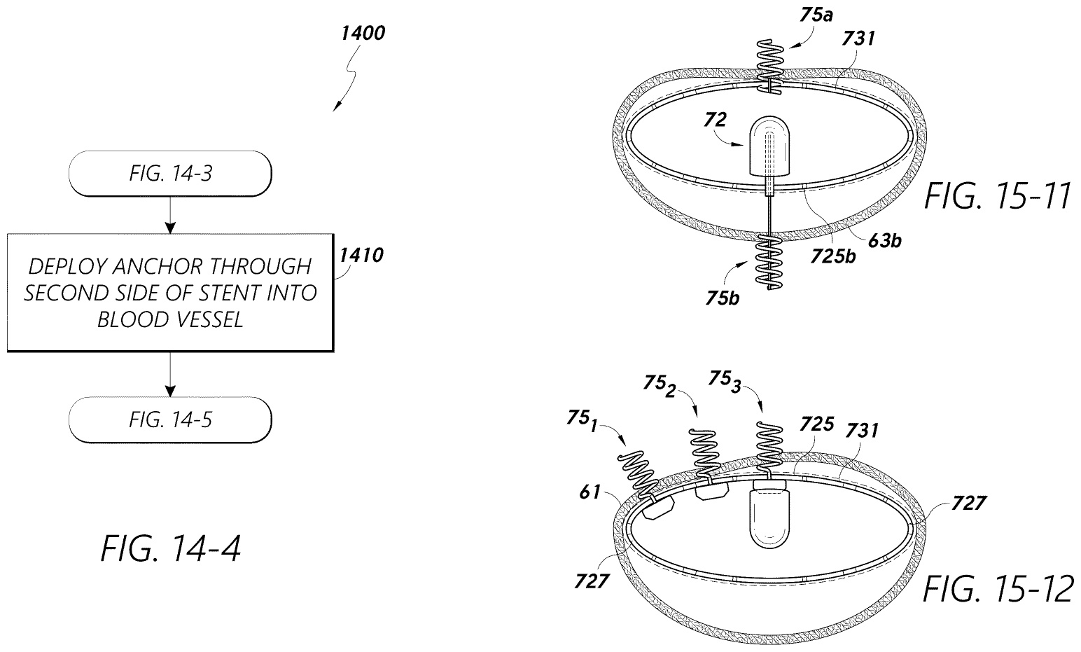

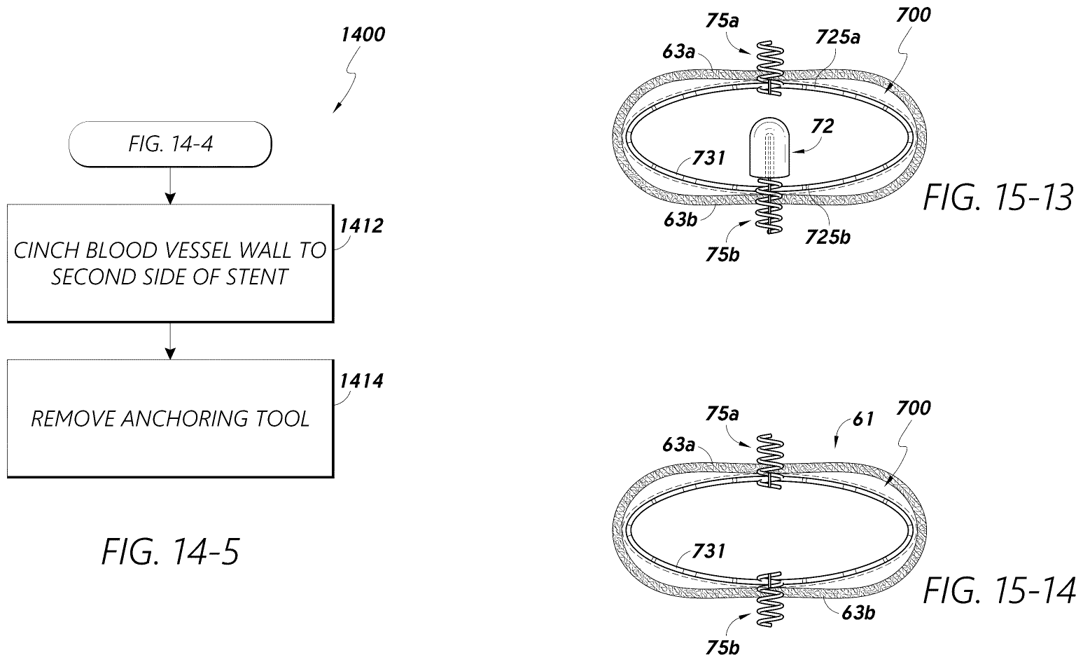

At block 1412, the process 1400 involves cinching the blood vessel wall to the second side 725b of the stent 700, as shown in FIG. 15-13. With the blood vessel 61 having been drawn proximate to both minor-axis sides 725 of the non-circular stent frame 731, the anchoring tool 72 may be removed, as in accordance with block 1414 of the process 1400 and shown in FIG. 15-14. With the blood vessel wall 61 secured to both minor-axis sidewalls 725 of the stent frame 731, reshaping of the stent frame 731 may produce commensurate reshaping in the blood vessel walls 63 that forces the blood vessel 61 to transition between more-circular and less-circular shapes, thereby restoring some amount of compliance functionality to the otherwise insufficiently-compliant blood vessel section 61.

Although certain images described above show two oppositely-directed tissue anchors, it should be understood that a series of anchors can be anchored to the blood vessel wall surrounding a stent in any desirable circumferential and/or axial arrangement with respect to the axis of the stent. The collection of tissue anchors may advantageously eliminate and/or reduce volume between the stent frame in the vascular wall, in which blood may otherwise be able to become trapped. In some implementations, a series of circumferentially-disposed tissue anchors are implemented to form multiple ring-like structures axially spaced from one another, or in a helical arrangement, or any other desired or appropriate arrangement. The plurality of tissue anchors deployed can be individually screwed/embedded and cinched around the stent frame, or tissue anchors can be first embedded in the blood vessel, after which deployed anchors may be cinched together and/or at the same time to effect the desired cinching of the blood vessel wall to the stent frame.

### ADDITIONAL EXAMPLES

Provided below is a list of examples, each of which may include aspects of any of the other examples disclosed herein. Furthermore, aspects of any example described above may be implemented in any of the numbered examples provided below.

Example 1: A non-circular stent comprising a stent frame comprising a first stent frame segment having a non-circular axial cross-sectional shape in a relaxed configuration thereof, the first stent frame segment including first and second minor-axis wall portions and first and second major-axis wall portions, and a plurality of tissue-engagement projections emanating from the first and second minor-axis wall portions and configured to couple the first and second minor-axis wall portions to a blood vessel wall.

Example 2: The non-circular stent of any example herein, in particular example 1, wherein the first and second major-axis wall portions do not have tissue-engagement projections associated therewith.

Example 3: The non-circular stent of any example herein, in particular example 1 or example 2, wherein the stent frame further comprises second and third stent frame segments disposed on opposite axial sides of the first stent frame segment, the second and third stent frame segments having a circular axial cross-sectional shape.

Example 4: The non-circular stent of any example herein, in particular example 3, wherein the second and third stent frame segments do not have tissue-engagement projections associated therewith.

Example 5: The non-circular stent of any example herein, in particular example 3 or example 4, further comprising a covering on at least one of an inner or outer diameter of the first stent frame segment.

Example 6: The non-circular stent of any example herein, in particular example 5, wherein the plurality of tissue-engagement projections puncture through the covering.

Example 7: The non-circular stent of any of any example herein, in particular any of examples 1-6, wherein the plurality of tissue-engagement projections project radially-outwardly with respect to an axis of the first stent frame segment.

Example 8: The non-circular stent of any example herein, in particular example 7, wherein the plurality of tissue-engagement projections deflect at an angle with respect to a dimension normal to a plane or surface of the non-circular stent.

Example 9: The non-circular stent of any of any example herein, in particular any of examples 1-8, wherein the plurality of tissue-engagement projections have retention features configured to impede withdrawal from tissue of the blood vessel wall when embedded therein.

Example 10: A method of coupling a stent frame to a blood vessel, the method comprising deploying a stent device in a blood vessel such that major-axis sides of a non-circular segment of the stent device contact an inner diameter of the blood vessel, and deflecting minor-axis walls of the non-circular segment of the stent device radially outward into contact with the inner diameter of the blood vessel, thereby causing tissue-engagement projections associated with the minor-axis walls to embed in the blood vessel.

Example 11: The method of any example herein, in particular example 10, further comprising, after said causing the tissue-engagement projections to embed in the blood vessel, pulling wall portions of the blood vessel radially inward by allowing the minor-axis walls to deflect radially inward.

Example 12: The method of any example herein, in particular example 10 example 11, wherein said deflecting the minor-axis walls of the non-circular segment of the stent device comprises circularizing the non-circular segment of the stent device.

Example 13: The method of any example herein, in particular example 12, wherein said circularizing the non-circular segment of the stent device is performed by inflating a balloon catheter within a channel of the non-circular segment of the stent device.

Example 14: The method of any example herein, in particular example 12 or example 13, wherein said circularizing the non-circular segment of the stent device is performed by deploying a circular stent within a channel of the non-circular segment of the stent device.

Example 15: The method of any of any example herein, in particular any of examples 10-14, wherein the major-axis sides do not have tissue-engagement projections associated therewith.

Example 16: The method of any of any example herein, in particular any of examples 10-15, wherein the stent device further comprises first and second circular stent segments disposed on opposite axial sides of the non-circular segment of the stent device.

Example 17: The method of any example herein, in particular example 16, wherein the first and second circular stent segments do not have tissue-engagement projections associated therewith.

Example 18: The method of any example herein, in particular example 16 or example 17, wherein the stent device comprises a covering on at least one of an inner or outer diameter of a stent frame of the stent device.

Example 19: The method of any example herein, in particular example 18, further comprising expelling trapped blood from a space between a portion of the covering associated with the non-circular segment and an inner diameter of the blood vessel.

Example 20: The method of any example herein, in particular example 19, wherein said deflecting the minor-axis walls of the non-circular segment of the stent device radially outward causes said expelling of the trapped blood.

Example 21: A non-circular stent assembly comprising a stent frame comprising a first stent frame segment having a non-circular axial cross-sectional shape in a relaxed configuration thereof, the first stent frame segment including first and second minor-axis wall portions and first and second major-axis wall portions, and a plurality of tissue anchors projecting radially outward from the first stent frame segment, the plurality of tissue anchors being secured to the first stent frame segment.

Example 22: The non-circular stent assembly of any example herein, in particular example 21, wherein the plurality of tissue anchors comprise helical tissue-engagement portions.

Example 23: The non-circular stent assembly of any example herein, in particular example 22, wherein the plurality of tissue anchors comprise drive heads configured to be rotated to cause rotation of the helical tissue-engagement portions.

Example 24: The non-circular stent assembly of any example herein, in particular example 23, wherein, when the plurality of tissue anchors are secured to the first stent frame segment, the drive heads are disposed within an inner diameter of the first stent frame segment.

Example 25: The non-circular stent assembly of any of any example herein, in particular any of examples 21-24, wherein each of the plurality of tissue anchors has a suture coupled thereto.

Example 26: The non-circular stent assembly of any example herein, in particular example 25, wherein the suture is configured to couple a respective one of the plurality of tissue anchors to the first stent frame segment.

Example 27: The non-circular stent assembly of any of any example herein, in particular any of examples 21-26, wherein the plurality of tissue anchors comprises a first tissue anchor coupled to the first minor-axis wall portion and a second tissue anchor coupled to the second minor-axis wall portion.

Example 28: The non-circular stent assembly of any of any example herein, in particular any of examples 21-27, wherein the stent frame has a fluid-impeding cover associated therewith.

Example 29: The non-circular stent assembly of any example herein, in particular example 28, wherein the stent frame includes second and third stent frame segments disposed on opposite axial sides of the first stent frame segment, the second and third stent frame segments having circular axial cross-sectional shapes.

Example 30: A method of coupling a stent frame to a blood vessel, the method comprising deploying a stent device in a blood vessel such that major-axis sides of a non-circular segment of the stent device contact an inner diameter of the blood vessel, passing at least a portion of a first tissue anchor through a first minor-axis wall of the non-circular segment, embedding a tip of the first tissue anchor in a wall of the blood vessel, and drawing the wall of the blood vessel towards the first minor-axis wall portion of the non-circular segment using the first tissue anchor.

Example 31: The method of any example herein, in particular example 30, wherein said passing the at least a portion of the first tissue anchor through the first minor-axis wall involves passing the first tissue anchor entirely through the first minor-axis wall.

Example 32: The method of any example herein, in particular example 31, wherein said drawing the wall of the blood vessel towards the first minor-axis wall portion involves pulling a line coupled to the first tissue anchor towards an inner channel of the stent device.

Example 33: The method of any of any example herein, in particular any of examples 30-32, wherein the first tissue anchor is a helical tissue anchor.

Example 34: The method of any of any example herein, in particular any of examples 30-33, wherein the first tissue anchor is a corkscrew tissue anchor comprising a helical tissue-engagement form and a drive head.

Example 35: The method of any example herein, in particular example 34, wherein said drawing the wall of the blood vessel towards the first minor-axis wall comprises rotating the drive head of the first tissue anchor from within an inner channel of the stent device.

Example 36: The method of any example herein, in particular example 35, further comprising deploying a driver tool from a catheter disposed within the inner channel of the stent device and engaging the driver tool with the drive head of the first tissue anchor within the inner channel of the stent device.

Example 37: The method of any of any example herein, in particular any of examples 30-36, further comprising passing at least a portion of a second tissue anchor through a second minor-axis wall portion of the non-circular segment, the second minor-axis wall portion being positioned opposite the first minor-axis wall portion, embedding a tip of the second tissue anchor in the wall of the blood vessel, and drawing the wall of the blood vessel towards the second minor-axis wall portion of the non-circular segment using the second tissue anchor.

Example 38: The method of any of any example herein, in particular any of examples 30-37, further comprising after said drawing the wall of the blood vessel towards the first minor-axis wall portion using the first tissue anchor, passing at least a portion of a second tissue anchor through the first minor-axis wall portion of the non-circular segment, embedding a tip of the second tissue anchor in the wall of the blood vessel, and drawing the wall of the blood vessel towards the first minor-axis wall portion using the second tissue anchor, and after said drawing the wall of the blood vessel towards the first minor-axis wall portion using the second tissue anchor, passing at least a portion of a third tissue anchor through the first minor-axis wall portion of the non-circular segment, embedding a tip of the third tissue anchor in the wall of the blood vessel, and drawing the wall of the blood vessel towards the first minor-axis wall portion using the third tissue anchor.

Example 39: The method of any example herein, in particular example 38, wherein the first, second, and third tissue anchors are passed through first, second, and third circumferential areas, respectively, of the first minor-axis wall portion.

Example 40: The method of any example herein, in particular example 39, wherein the first circumferential area of the first minor-axis wall portion is closer to one of the major-axis sides of the non-circular segment than the second and third circumferential areas are to either of the major-axis sides.

Example 41: The method of any example herein, in particular example 40, wherein the third circumferential area of the first minor-axis wall portion is closer to a minor axis of the non-circular segment than both the first and second circumferential areas are.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method of coupling a non-circular implant within a blood vessel for increasing blood flow through the blood vessel, the method comprising:
deploying a stent device in a blood vessel such that major-axis sides of a non-circular segment of the stent device contact an inner diameter of the blood vessel;
passing at least a portion of a first tissue anchor through a first minor-axis wall portion of the non-circular segment;
embedding a tip of the first tissue anchor in a wall of the blood vessel; and
drawing the wall of the blood vessel towards the first minor-axis wall portion of the non-circular segment using the first tissue anchor.

2. The method of claim 1, wherein passing the at least a portion of the first tissue anchor through the first minor-axis wall portion involves passing the first tissue anchor entirely through the first minor-axis wall portion.

3. The method of claim 2, wherein drawing the wall of the blood vessel towards the first minor-axis wall portion involves pulling a line coupled to the first tissue anchor towards an inner channel of the stent device.

4. The method of claim 1, wherein the first tissue anchor is a helical tissue anchor.

5. The method of claim 1, wherein the first tissue anchor is a corkscrew tissue anchor comprising a helical tissue-engagement form and a drive head.

6. The method of claim 5, wherein drawing the wall of the blood vessel towards the first minor-axis wall portion comprises rotating the drive head of the first tissue anchor from within an inner channel of the stent device.

7. The method of claim 6, further comprising deploying a driver tool from a catheter disposed within the inner channel of the stent device and engaging the driver tool with the drive head of the first tissue anchor within the inner channel of the stent device.

8. The method of claim 1, further comprising:
passing at least a portion of a second tissue anchor through a second minor-axis wall portion of the non-circular segment, the second minor-axis wall portion being positioned opposite the first minor-axis wall portion;
embedding a tip of the second tissue anchor in the wall of the blood vessel; and
drawing the wall of the blood vessel towards the second minor-axis wall portion of the non-circular segment using the second tissue anchor.

9. The method of claim 1, further comprising:
after drawing the wall of the blood vessel towards the first minor-axis wall portion using the first tissue anchor:
passing at least a portion of a second tissue anchor through the first minor-axis wall portion of the non-circular segment;
embedding a tip of the second tissue anchor in the wall of the blood vessel; and
drawing the wall of the blood vessel towards the first minor-axis wall portion using the second tissue anchor; and
after drawing the wall of the blood vessel towards the first minor-axis wall portion using the second tissue anchor:
passing at least a portion of a third tissue anchor through the first minor-axis wall portion of the non-circular segment;
embedding a tip of the third tissue anchor in the wall of the blood vessel; and
drawing the wall of the blood vessel towards the first minor-axis wall portion using the third tissue anchor.

10. The method of claim 9, wherein;
the first, second, and third tissue anchors are passed through first, second, and third circumferential areas, respectively, of the first minor-axis wall portion; and
the first circumferential area of the first minor-axis wall portion is closer to one of the major-axis sides of the non-circular segment than the second and third circumferential areas are to either of the major-axis sides.

11. A method of coupling a non-circular stent frame to a blood vessel, the method comprising:
deploying a stent device in a blood vessel, the stent device comprising a stent frame forming first and second circular axial end segments and a non-circular medial segment having a minor-axis diameter and a major-axis diameter that is greater than the minor-axis diameter;
pressing major-axis end walls of the medial segment of the frame against an inner surface of the blood vessel to cause ovalization of the blood vessel;
outwardly deflecting minor-axis walls of the stent frame to press the minor-axis walls against the inner surface of the blood vessel to thereby embed a plurality of tissue-engagement barbs that project from struts of the stent frame into the inner surface of the blood vessel to couple the minor-axis walls to the blood vessel; and
cyclically reshaping the blood vessel between circular and less-circular shapes by allowing the medial segment of the stent frame to cyclically shorten and lengthen the major-axis diameter in response to systolic pressure in the blood vessel over multiple cardiac cycles, thereby smoothing a pressure waveform associated with the blood vessel.

12. The method of claim 11, wherein the plurality of tissue-engagement barbs project in-plane with a plane of the stent frame in the medial segment.

13. The method of claim 12, wherein at least some struts of the stent frame in the medial segment have tissue-engagement barbs projecting from both axial sides thereof.

14. The method of claim 11, wherein tissue-engagement barbs are associated only with the minor-axis walls of the stent frame and not with the major-axis end walls of the medial segment or the first and second axial end segments of the stent frame.

15. The method of claim 11, wherein the stent device further comprises a membrane covering on at least one of an inner or outer diameter of the medial segment of the stent frame.

16. The method of claim 15, wherein the plurality of tissue-engagement barbs puncture through the membrane covering.

17. The method of claim 11, wherein the plurality of tissue-engagement barbs project radially-outwardly with respect to an axis of the stent frame at an acute angle with respect to the axis.

18. The method of claim 11, wherein the plurality of tissue-engagement barbs have retention features configured to impede withdrawal from tissue of the blood vessel when embedded therein.

19. A method of coupling a non-circular stent frame to a blood vessel, the method comprising:
deploying a stent device in a blood vessel such that major-axis sides of a non-circular segment of the stent device contact an inner wall of the blood vessel;
passing at least a portion of a corkscrew tissue anchor through a first minor-axis wall portion of the non-circular segment;
embedding a tip of the corkscrew tissue anchor in the inner wall of the blood vessel; and
securing the inner wall of the blood vessel against the first minor-axis wall portion of the non-circular segment by rotating the corkscrew tissue anchor.

20. The method of claim 19, wherein securing the inner wall of the blood vessel against the first minor-axis wall portion involves rotating a tissue anchor head positioned within an inner lumen of the non-circular segment of the stent device.
